(19) United States
(12) Patent Application Publication
(10) Pub. No.: US 2023/0167496 A1
(43) Pub. Date: Jun. 1, 2023

(54) COMPOSITIONS AND METHODS FOR ISOLATING NUCLEIC ACID MOLECULES

(71) Applicant: 10x Genomics, Inc., Pleasanton, CA (US)

(72) Inventor: Felice Alessio BAVA, Rome (IT)

(73) Assignee: 10x Genomics, Inc., Pleasanton, CA (US)

(21) Appl. No.: 17/957,781

(22) Filed: Sep. 30, 2022

Related U.S. Application Data
(60) Provisional application No. 63/251,252, filed on Oct. 1, 2021.

(51) Int. Cl. C12Q 1/6874; C12N 15/10

(52) U.S. Cl. CPC C12Q 1/6874; C12N 15/1065

(57) ABSTRACT

This invention provides methods, compositions, kits and systems for isolating and/or enriching nucleic acid molecules. In some aspects, this disclosure provides methods for enriching nucleic acid molecules that can be used for transcriptomics. Methods of this invention can detect nucleic acid molecules of a sample that may be masked by highly expressed molecules.

### TECHNICAL FIELD

This invention relates to methods, compositions, kits and systems for selectively isolating and/or enriching a molecule in a mixture or library of molecules. More particularly, this disclosure provides methods for isolating and/or enriching a nucleic acid molecule, such as an RNA, in a diverse population of nucleic acid molecules.

### BACKGROUND

The study of complex biology, particularly with regard to human health and disease, can utilize transcriptomic analyses on a massive scale. Whole genome or whole transcriptome analyses can impose significant costs. One way to reduce the costs associated with massively parallel sequencing while retaining the benefits of large scale analysis is to perform high throughput, high accuracy sequencing on targeted regions of the genome or transcriptome. Targeted sequencing can be achieved by enriching nucleic acid libraries for targets of interest, e.g., via hybridization capture of library members representing targets of interest and pulldown of the captured members.

Significant drawbacks in hybridization capture methods can include the need to provide capture molecules which specifically bind to a large number of target regions of interest and to modify the large number of capture molecules, e.g., with a bait molecule for pulldown. Complex preparation schemes may thus be required to cover all the regions of interest.

What is needed are methods, compositions and systems for isolating and/or enriching nucleic acid molecules of interest. It is also desirable to broadly detect, sequence and represent target regions and transcript species with high specificity and efficiency.

There is a need for methods for isolating target nucleic acid molecules for transcriptomics. What is needed are methods and systems that provide efficient capture of target nucleic acid molecules of interest with increased affinity, as well as utilize capture molecules or moieties that are not based on the very same targets or molecules of interest.

### BRIEF SUMMARY

This invention provides methods, compositions, kits and systems for processing, isolating and/or enriching nucleic acid molecules. In some aspects, this disclosure provides methods for enriching nucleic acid molecules that can be used for transcriptomics. Methods of this invention can detect nucleic acid molecules of a sample that may be masked by highly expressed molecules.

Embodiments of this invention provide methods and compositions for processing, isolating or enriching nucleic acid molecules of interest. Nucleic acid molecules of interest can be detected with reduced DNA contamination.

In certain aspects, methods and compositions of this disclosure can be used to detect a wide variety of different samples containing nucleic acid molecules from different compartments. The methods and compositions of this invention can be used to reduce the presence of unprocessed or partially processed nucleic acid molecules and enhance the determination of molecules of interest.

In some embodiments, this invention provides methods and compositions for transcriptome sequencing with efficient capture molecules that specifically bind a large number of target regions of interest. In certain embodiments, the efficient capture molecules may have sequences that do not derive from any target of interest. The efficient capture molecules may reduce the amount of sequencing work needed to detect target regions of interest.

In further aspects, the methods, compositions and systems of this disclosure for processing, isolating and/or enriching nucleic acid molecules of interest can be used to broadly detect, sequence and represent target regions and transcript species with high specificity and efficiency.

In additional embodiments, the methods, compositions and systems of this invention can be used for processing, isolating and/or enriching target nucleic acid molecules for transcriptomics. This invention can provide efficient capture of target nucleic acid molecules of interest.

Embodiments of this invention include:

A method for processing one or more nucleic acid target molecules in a mixture of nucleic acid molecules, the method comprising: contacting the mixture with one or more primary oligonucleotides which hybridize to the nucleic acid target molecules, wherein each of the primary oligonucleotides comprises one or more target analyte capture sequences and one or more universal capture sequences; contacting the mixture with one or more secondary oligonucleotides which hybridize to the primary oligonucleotides, wherein each of the secondary oligonucleotides comprises one or more universal sequences which hybridize to the universal capture sequences of the primary oligonucleotides.

A method for isolating one or more nucleic acid target molecules in a mixture of nucleic acid molecules, the method comprising: contacting the mixture with one or more primary oligonucleotides which hybridize to the nucleic acid target molecules, wherein each of the primary oligonucleotides comprises one or more target analyte capture sequences and one or more universal capture sequences; contacting the mixture with one or more secondary oligonucleotides which hybridize to the primary oligonucleotides, wherein each of the secondary oligonucleotides comprises one or more universal sequences which hybridize to the universal capture sequences of the primary oligonucleotides; and isolating the secondary oligonucleotides, which are attached to the primary oligonucleotides and the nucleic acid target molecules, using one or more bait molecules attached to each of the secondary oligonucleotides, the bait molecules having binding affinity to one or more binding partners.

A method for enriching one or more nucleic acid target molecules in a mixture of nucleic acid molecules, the method comprising: contacting the mixture with one or more primary oligonucleotides which hybridize to the nucleic acid target molecules, wherein each of the primary oligonucleotides comprises one or more target analyte capture sequences and one or more universal capture sequences; contacting the mixture with one or more secondary oligonucleotides which hybridize to the primary oligonucleotides, wherein each of the secondary oligonucleotides comprises one or more universal sequences which hybridize to the universal capture sequences of the primary oligonucleotides; isolating the secondary oligonucleotides, which are attached to the primary oligonucleotides and the nucleic acid target molecules, using one or more bait molecules attached to each of the secondary oligonucleotides, the bait molecules having binding affinity to one or more binding partners; and removing nucleic acid molecules from the mixture that are not attached to the primary oligonucleotide.

The method above, wherein the mixture is a library of nucleic acid molecules.

The method above, wherein the mixture is a RNA, DNA, or cDNA library.

The method above, wherein the mixture is a barcoded library.

The method above, wherein the mixture is a barcoded nucleic acid library.

The method above, wherein the nucleic acid target molecule is barcoded with one or more barcode sequences.

The method above, wherein the nucleic acid target molecule comprises one or more UMI sequences.

The method above, wherein the nucleic acid target molecules comprise one or more portions of one or more functional sequences.

The method above, wherein the target analyte capture sequences are contiguous.

The method above, wherein the target analyte capture sequences hybridize to one or more sequences of the nucleic acid target molecules.

The method above, wherein the target analyte capture sequences are about 5-500 nucleotides in length.

The method above, wherein the target analyte capture sequences are about 15-30 nucleotides in length.

The method above, wherein the universal capture sequences are contiguous.

The method above, wherein the universal capture sequences are about 5-500 nucleotides in length.

The method above, wherein the universal capture sequences are about 15-30 nucleotides in length.

The method above, wherein the universal capture sequences are not present in any genome associated with the nucleic acid target molecules.

The method above, wherein the universal capture sequences are not present in any molecule in the mixture of nucleic acid molecules.

The method above, wherein the universal capture sequences comprise, or are complementary to, any contiguous sequence of a T7 promoter.

The method above, wherein each of the universal capture sequences comprises one or more locked nucleic acid monomers.

The method above, wherein each of the universal capture sequences comprises one or more chemically-modified nucleotides.

The method above, wherein each of the universal capture sequences comprises one or more chemically-modified 2′-OMe nucleotides.

The method above, wherein the target analyte capture sequences and the universal capture sequences of each of the primary oligonucleotides are within a proximity of 30 nucleotides.

The method above, wherein the target analyte capture sequences and the universal capture sequences of each of the primary oligonucleotides are within a proximity of five nucleotides.

The method above, wherein the nucleic acid target molecules each comprise two or more target analyte sequences, wherein each target analyte sequence hybridizes to a target analyte capture sequence of the primary oligonucleotides, and wherein adjacent target analyte sequences of each of the nucleic acid target molecules are within a proximity of 5-30 nucleotides.

The method above, wherein the nucleic acid target molecules each comprise two or more target analyte sequences, wherein each target analyte sequence hybridizes to a target analyte capture sequence of the primary oligonucleotides, and wherein adjacent target analyte sequences of each of the nucleic acid target molecules are within a proximity of five nucleotides.

The method above, wherein each of the primary oligonucleotides hybridizes to one or more target analyte sequences of the nucleic acid target molecules, and wherein each of the primary oligonucleotides comprises the same universal capture sequence.

The method above, wherein each of the primary oligonucleotides hybridizes to one or more target analyte sequences of the nucleic acid target molecules, and wherein each of the primary oligonucleotides comprises two or more different universal capture sequences.

The method above, wherein each of the secondary oligonucleotides comprises a plurality of the same universal sequence.

The method above, wherein each of the secondary oligonucleotides comprises a plurality of the same universal sequence, and wherein the plurality of universal sequences are spaced apart by one or more nucleotides on the secondary oligonucleotides.

The method above, wherein each of the secondary oligonucleotides comprises a plurality of the same universal sequence, and wherein the plurality of universal sequences are spaced apart by one or more nucleotides on the secondary oligonucleotides, so that adjacent universal sequences are within a proximity of five nucleotides.

The method above, wherein the secondary oligonucleotides each comprise a single universal sequence, and the secondary oligonucleotides collectively comprise a plurality of different universal sequences.

The method above, wherein the primary oligonucleotides each comprise a single target analyte capture sequence, and wherein the primary oligonucleotides collectively hybridize to a plurality of different target analyte sequences.

The method above, wherein the secondary oligonucleotides each comprise one or more attached bait molecules having binding affinity to a binding partner.

The method above, wherein the secondary oligonucleotides each comprise one or more attached bait molecules having binding affinity to a binding partner, and wherein the binding partner is attached to a solid support.

The method above, wherein the secondary oligonucleotides each comprise one or more attached bait molecules having binding affinity to a binding partner, and wherein the binding partner is attached to a solid support comprising a bead, a magnetic bead, or a paramagnetic bead, optionally wherein the method further comprises isolating the bead, magnetic bead, or paramagnetic bead.

The method above, wherein the bait molecule has specific binding affinity for the binding partner.

The method above, wherein the bait molecule is biotin and wherein the binding partner is streptavidin.

A method for processing one or more nucleic acid target molecules in a mixture of nucleic acid molecules, the method comprising: contacting the mixture with two or more primary oligonucleotides which hybridize to the nucleic acid target molecules, wherein each of the primary oligonucleotides comprises one target analyte capture sequence and one universal capture sequence, and wherein the target analyte capture sequences of the primary oligonucleotides are different, and wherein the universal capture sequences of the primary oligonucleotides are the same; contacting the mixture with secondary oligonucleotides which hybridize to the primary oligonucleotides, wherein each of the secondary oligonucleotides comprises two or more universal sequences which hybridize to the universal capture sequences of the primary oligonucleotides, and wherein the universal sequences of the secondary oligonucleotides are the same.

The method above, wherein the primary oligonucleotides are hybridized to the nucleic acid target molecules within a proximity of 5 to 30 nucleotides.

A kit for processing, isolating or enriching one or more nucleic acid target molecules in a mixture of nucleic acid molecules, the kit comprising one or more primary oligonucleotides which hybridize to one or more nucleic acid target molecules, wherein the primary oligonucleotides each comprise one or more target analyte capture sequences and one or more universal capture sequences.

The kit above, further comprising one or more secondary oligonucleotides which hybridize to the primary oligonucleotides, wherein each of the secondary oligonucleotides comprises one or more sequences which hybridize to the universal capture sequences of the primary oligonucleotides, and wherein each of the secondary oligonucleotides comprises one or more attached bait molecules having binding affinity to a binding partner.

A nucleic acid complex, comprising a nucleic acid target molecule hybridized to two or more primary oligonucleotides, wherein the nucleic acid target molecule comprises one or more target analyte sequences, wherein each of the primary oligonucleotides comprises one or more target analyte capture sequences and one or more universal capture sequences, wherein the target analyte capture sequences hybridize to the target analyte sequences, wherein one or more of the primary oligonucleotides is hybridized to a secondary oligonucleotide, and wherein each of the secondary oligonucleotides comprises one or more universal sequences which hybridize to the universal capture sequences of the primary oligonucleotides.

The complex above, wherein the complex is a multi-strand structure.

The complex above, wherein each of the secondary oligonucleotides comprises a bait molecule having binding affinity to a binding partner.

### BRIEF DESCRIPTION OF THE DRAWINGS

In the drawings and descriptions herein, a lower case letter (e.g., “a” or “a′”) can represent a specific nucleotide in a sequence of nucleotides. An apostrophe after a lower case letter (e.g., “a′”) indicates that the nucleotide represented by the letter with the apostrophe is complementary to a nucleotide represented by the same letter without an apostrophe. For example, “a” is complementary to “a′.” As used herein, complementarity refers to sequences that can hybridize to one another.

FIG. 1A and FIG. 1B illustrate an embodiment for processing, isolating and/or enriching nucleic acid molecules 100. In FIG. 1A, a target analyte 110 is illustrated. The target analyte 110 may be a nucleic acid. In some examples, the target analyte 110 may be a cDNA. The target analyte 110 may have a nucleotide sequence, represented by “abcd” which may be called a target analyte sequence 111. Also illustrated in FIG. 1A is a primary (1°) probe 120. The 1° probe 120 may be a nucleic acid. The 1° probe 120 may have a target analyte capture sequence 121, represented by “a′b′c′d′.” The target analyte capture sequence 121 in the 1° probe 120 can be complementary to the target analyte sequence 111 in the target analyte 110. The target analyte capture sequence 121 and the target analyte sequence 111 can be capable of hybridizing to one another. The 1° probe 120 may have a universal capture sequence 122, represented by “efgh” in FIG. 1A. Also illustrated in FIG. 1A is a secondary (2°) probe 160. The 2° probe 160 may be a nucleic acid. The 2° probe 160 may have a universal sequence 162, represented by “e′f′g′h′,” which may be referred to herein as a 1° probe capture sequence. The universal sequence 162 in the 2° probe 160 can be complementary to the universal capture sequence 122 in the 1° probe 120. The universal sequence 162 and the universal capture sequence 122 can be capable of hybridizing to one another. The 2° probe 160 may have an attached bait molecule 169. In some examples, the bait molecule 169 may be biotin. FIG. 1B shows an operational embodiment having the target analyte 110 bound to the 1° probe 120, which in turn is bound to the 2° probe 160.

FIG. 2A and FIG. 2B illustrate an embodiment 200 for processing, isolating and/or enriching nucleic acid molecules. In FIG. 2A, a target analyte 210 is illustrated, which may have a target analyte sequence 211 represented by “abcd.” FIG. 2A shows a primary (1°) probe 220, which may have a target analyte capture sequence 221, represented by “a′b′c′d′.” The 1° probe 220 may have a universal capture sequence 222, represented by “efgh.” FIG. 2A shows a secondary (2°) probe 260, which may have a universal sequence 262 represented by “h′g′f′e′.” The 2° probe 260 may have an attached bait molecule 269. FIG. 2B shows an operational embodiment having the target analyte 210 bound to the 1° probe 220, which in turn is bound to the 2° probe 260.

FIG. 3A and FIG. 3B illustrate an embodiment 500 for processing, isolating and/or enriching nucleic acid molecules. In FIG. 3A, a target analyte 510 is illustrated, which may have a first target analyte sequence 511 represented by “abcd” and a second target analyte sequence 513 represented by “ijkl.” FIG. 3A shows a primary (1°) probe 520, which may have a target analyte capture sequence 521, represented by “a′b′c′d′.” The 1° probe 520 may have a universal capture sequence 522, represented by “efgh.” FIG. 3A shows an additional primary (1°) probe 530, which may have a target analyte capture sequence 533, represented by “i′j′k′l′.” The 1° probe 530 may have a universal capture sequence 522, represented by “efgh.” FIG. 3A shows a secondary (2°) probe 560, which may have a 1° probe capture sequence 562 represented by “e′f′g′h′.” The 2° probe 560 may have an attached bait molecule 569. FIG. 3B shows an operational embodiment having the target analyte 510 bound to the 1° probes 520 and 530, which in turn are bound to 2° probes 560. The universal capture sequence of the 1° probes 520 and 530 may be the same. A bait molecule may be attached to each of the 2° probes.

FIG. 4A and FIG. 4B illustrate an embodiment 600 for processing, isolating and/or enriching nucleic acid molecules. In FIG. 4A, a target analyte 610 is illustrated, which may have a first target analyte sequence 611 represented by “abcd” and a second target analyte sequence 613 represented by “ijkl.” FIG. 4A shows a primary (1°) probe 620, which may have a target analyte capture sequence 621, represented by “a′b′c′d′.” The 1° probe 620 may have a universal capture sequence 622, represented by “efgh.” FIG. 4A shows an additional primary (1°) probe 630, which may have a target analyte capture sequence 633, represented by “i′j′k′l′.” The 1° probe 630 may have a universal capture sequence 634, represented by “mnop.” The universal capture sequence 622 of primary (1°) probe 620 and the universal capture sequence 634 of 1° probe 630 may be different. FIG. 4A shows a secondary (2°) probe 660, which may have a 1° probe capture sequence 662 represented by “e′f′g′h′.” The 2° probe 660 may have an attached bait molecule 669. FIG. 4A shows an additional secondary (2°) probe 670, which may have a 1° probe capture sequence 674 represented by “m′n′o′p′.” The 2° probe 670 may have an attached bait molecule 679. FIG. 4B shows an operational embodiment having the target analyte 610 bound to the 1° probes 620 and 630, which are each in turn bound to 2° probes 660 and 670.

FIG. 5A and FIG. 5B illustrate an embodiment 700 for processing, isolating and/or enriching nucleic acid molecules. In FIG. 5A, a target analyte 710 is illustrated, which may have a first target analyte sequence 711 represented by “abcd” and a second target analyte sequence 713 represented by “ijkl.” FIG. 5A shows a primary (1°) probe 720, which may have a target analyte capture sequence 721, represented by “a′b′c′d′.” The 1° probe 720 may have a universal capture sequence 722, represented by “efgh.” FIG. 5A shows an additional primary (1°) probe 730, which may have a target analyte capture sequence 733, represented by “i′j′k′l′.” The 1° probe 730 may have a universal capture sequence 732, represented by “efgh.” FIG. 5A shows a secondary (2°) probe 760, which may have two or more 1° probe capture sequences 762 represented by “e′f′g′h′.” The 2° probe 760 may have an attached bait molecule 769.

FIG. 5B shows an operational embodiment having the target analyte 710 bound to the 1° probes 720 and 730, which are each in turn bound to 2° probe 760. Because in these embodiments the 2° probe attaches to both primary probes, the analyte can be bound in a multi-strand structure.

FIG. 5B shows an operational embodiment having the target analyte 710 bound to the 1° probes 720 and 730, which are each in turn bound to 2° probe 760. In these embodiments, the 2° probe can attach to both primary probes, so that the analyte can be bound with greater affinity than when the 2° probe attaches to only one primary probe. In these embodiments, the analyte can be advantageously bound with greater affinity, even under more stringent conditions such as higher temperatures, lower salt, and/or presence of organic solvent.

The locked, multi-strand superstructure can be formed when the first and second target sequences 711 and 713 are in proximity to each other, the target analyte capture sequence 721 is in proximity to the universal capture sequence 722 of the same primary probe, the target analyte capture sequence 733 is in proximity to the universal capture sequence 732 of the same primary probe, and the 1° probe capture sequences 762 are in proximity to each other.

FIG. 6A and FIG. 6B illustrate an embodiment 800 for processing, isolating and/or enriching nucleic acid molecules. In FIG. 6A, a target analyte 810 is illustrated, which may have a first target analyte sequence 811 represented by “abcd” and a second target analyte sequence 813 represented by “ijkl.” FIG. 6A shows a primary (1°) probe 820, which may have a target analyte capture sequence 821, represented by “a′b′c′d′.” The 1° probe 820 may have a universal capture sequence 822, represented by “efgh.” FIG. 6A shows an additional primary (1°) probe 830, which may have a target analyte capture sequence 833, represented by “i′j′k′l′.” The 1° probe 830 may have a universal capture sequence 834, represented by “mnop.” FIG. 6A shows a secondary (2°) probe 860, which may have two or more 1° probe capture sequences 862 represented by “e′f′g′h′” and 864 represented by “m′n′o′p′.” The 2° probe 860 may have an attached bait molecule 869. FIG. 6B shows an operational embodiment having the target analyte 810 bound to the 1° probes 820 and 830, which are each in turn bound to 2° probe 860.

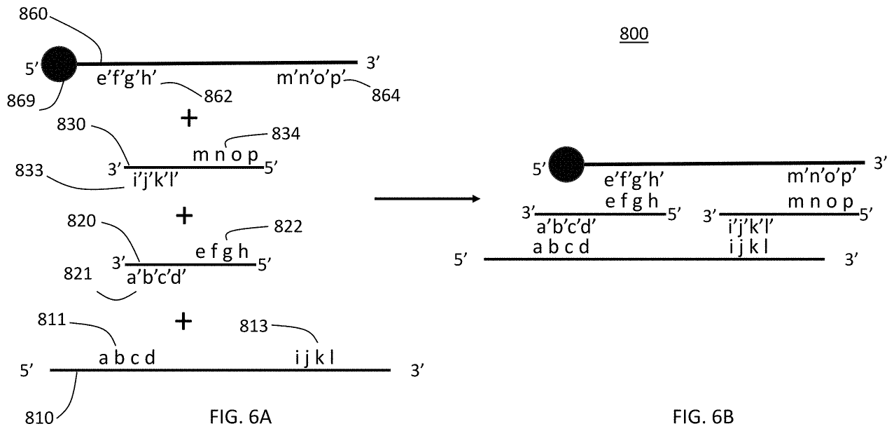

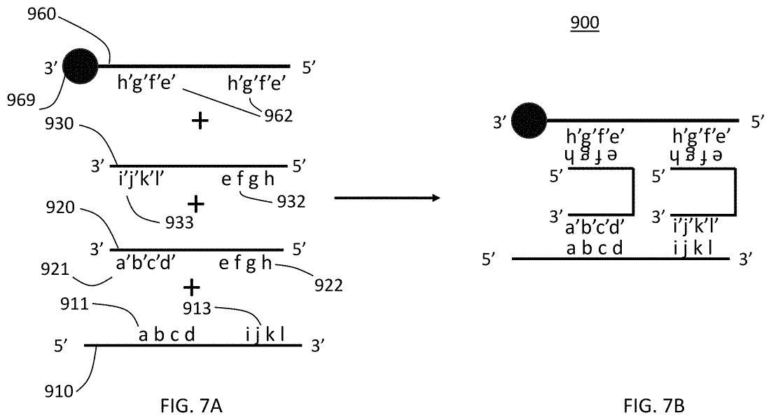

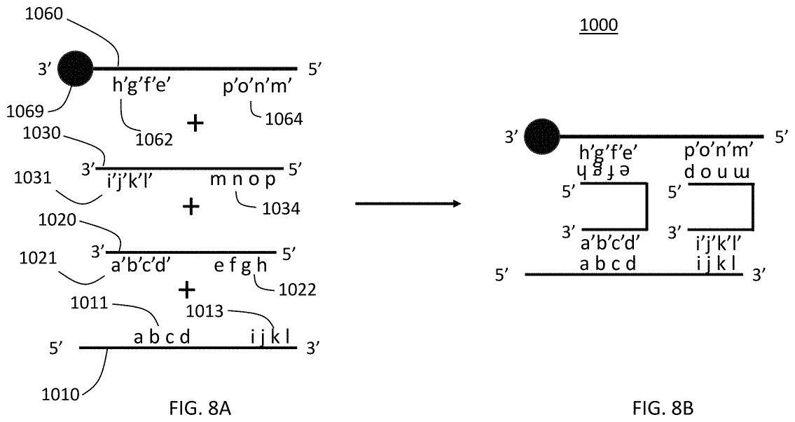

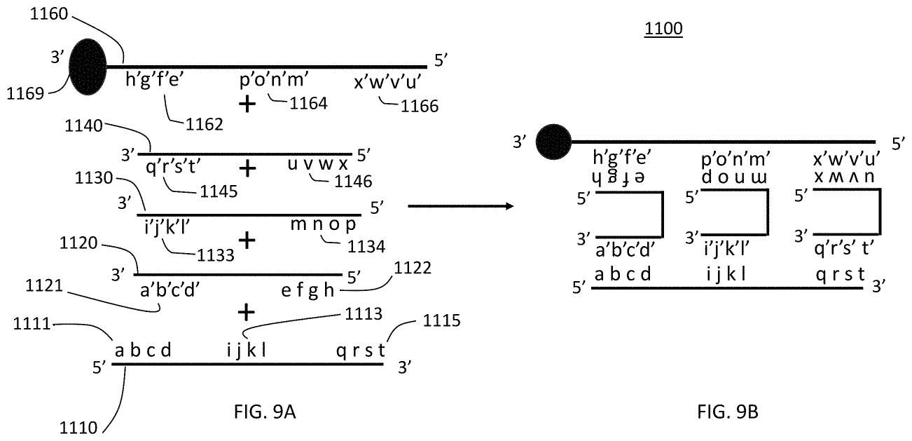

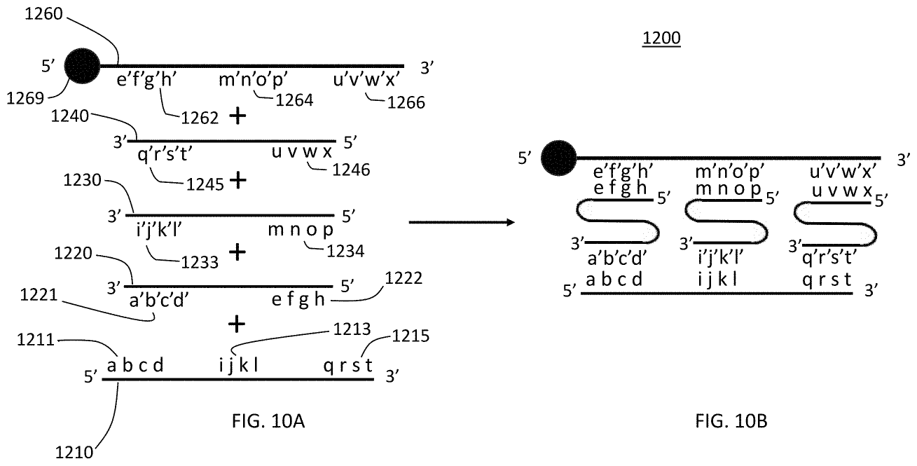

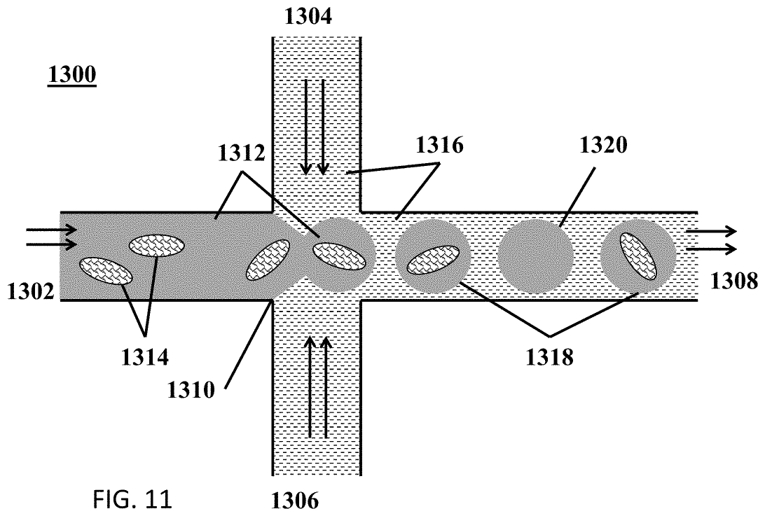

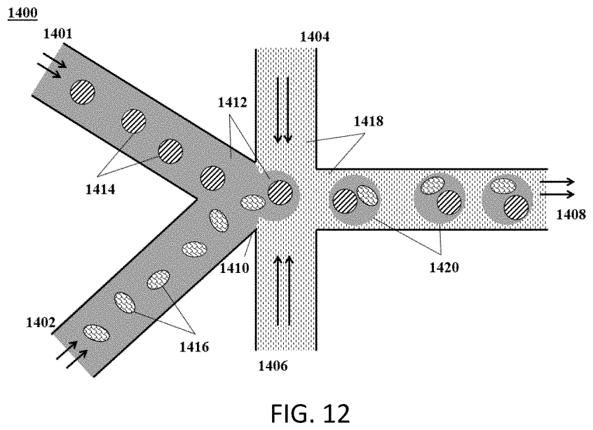

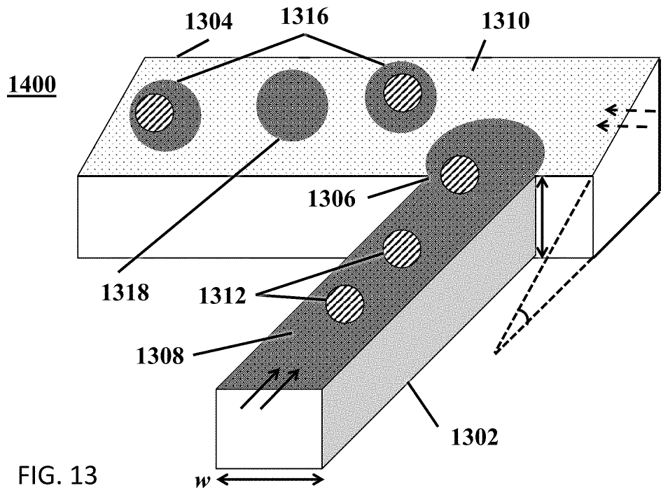

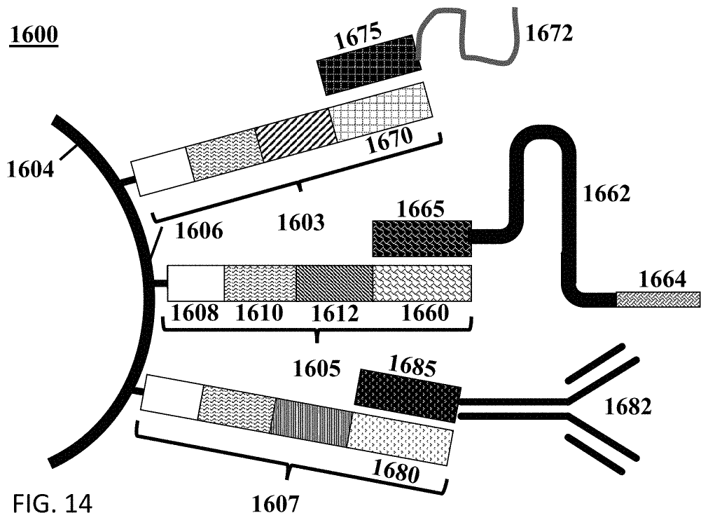

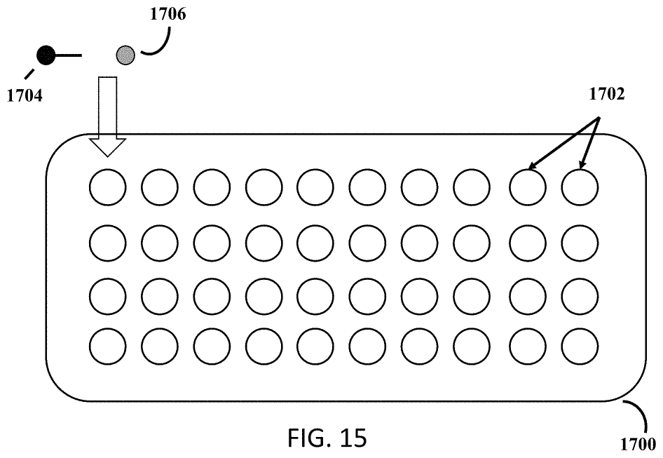

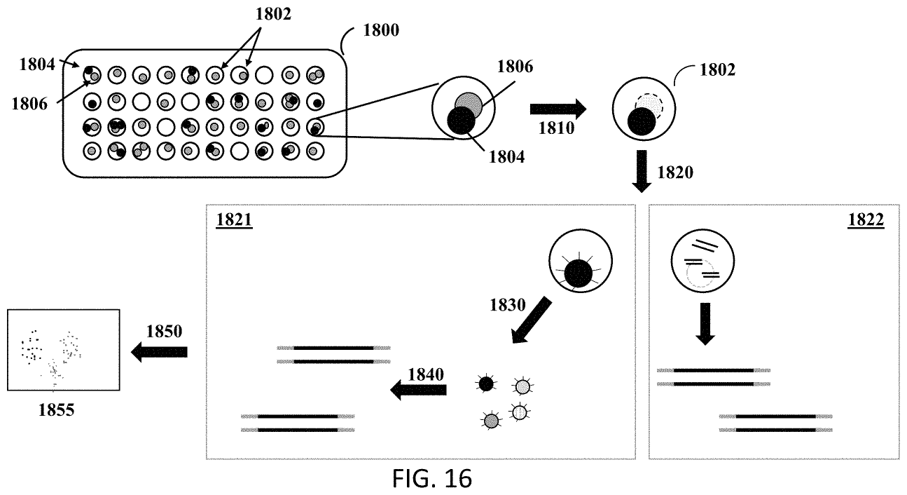

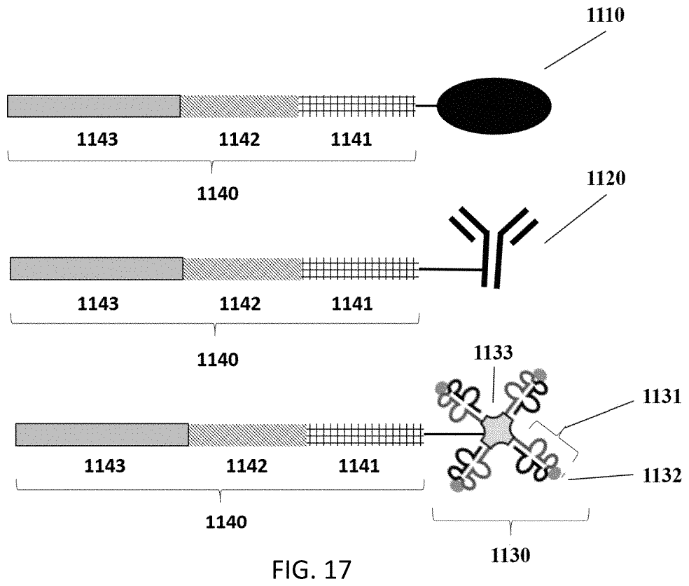

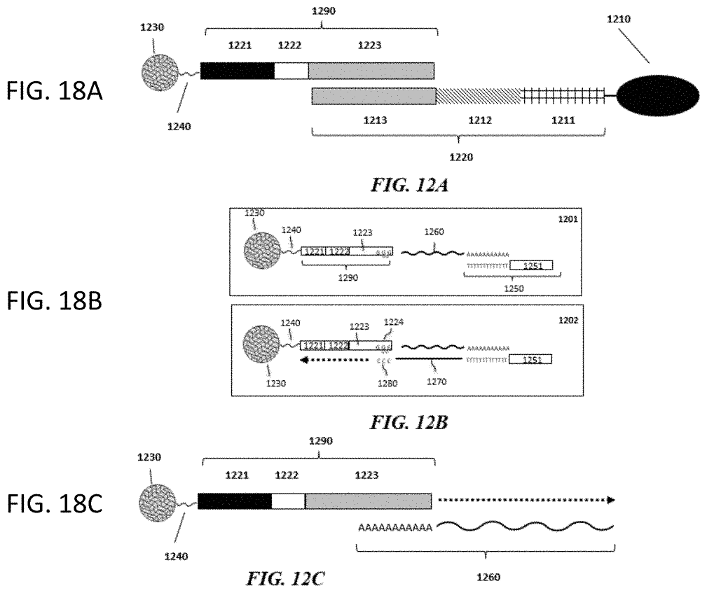

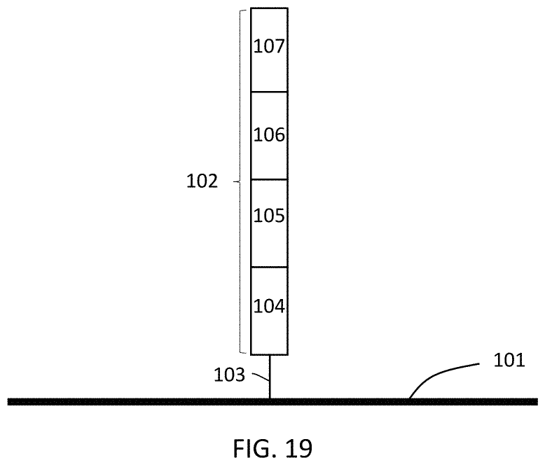

Referring to FIG. 6A and FIG. 6B, because the 2° probe attaches to both primary probes, in some embodiments, the analyte can be bound in a locked, multi-strand superstructure. The locked, multi-strand superstructure can be formed when the first and second target sequences 810 and 813 are in proximity to each other, the target analyte capture sequence 821 is in proximity to the universal capture sequence 822 of the same primary probe, the target analyte capture sequence 833 is in proximity to the universal capture sequence 834 of the same primary probe, and the 1° probe capture sequences 862 and 864 are in proximity to each other.

FIG. 6B shows an operational embodiment having the target analyte 810 bound to the 1° probes 820 and 830, which are each in turn bound to 2° probe 860. In these embodiments, the 2° probe can attach to both primary probes, so that the analyte can be bound with greater affinity than when the 2° probe attaches to only one primary probe. In these embodiments, the analyte can be advantageously bound with greater affinity, even under more stringent conditions such as higher temperatures, lower salt, and/or presence of organic solvent.

FIG. 7A and FIG. 7B illustrate an embodiment 900 for processing, isolating and/or enriching nucleic acid molecules. In FIG. 7A, a target analyte 910 is illustrated, which may have a first target analyte sequence 911 represented by “abcd” and a second target analyte sequence 913 represented by “ijkl.” FIG. 7A shows a primary (1°) probe 920, which may have a target analyte capture sequence 921, represented by “a′b′c′d′.” The 1° probe 920 may have a universal sequence 922, represented by “efgh.” FIG. 7A shows an additional primary (1°) probe 930, which may have a target analyte capture sequence 933, represented by “i′j′k′l′.” The 1° probe 930 may have a universal sequence 932, represented by “efgh.” FIG. 7A shows a secondary (2°) probe 960, which may have two or more 1° probe capture sequences 962 each represented by “h′g′f′e′.” The 2° probe 960 may have an attached bait molecule 969. FIG. 7B shows an operational embodiment having the target analyte 910 bound to the 1° probes 920 and 930, which are each in turn bound to 2° probe 960.

Referring to FIG. 7A and FIG. 7B, because the 2° probe attaches to both primary probes, in some embodiments, the analyte can be bound in a locked, multi-strand superstructure. The locked, multi-strand superstructure can be formed when the first and second target sequences 911 and 913 are in proximity to each other, the target analyte capture sequence 921 is in proximity to the universal capture sequence 922 of the same primary probe, the target analyte capture sequence 933 is in proximity to the universal capture sequence 932 of the same primary probe, and the 1° probe capture sequences 962 are in proximity to each other.

FIG. 7B shows an operational embodiment having the target analyte 910 bound to the 1° probes 920 and 930, which are each in turn bound to 2° probe 960. In these embodiments, the 2° probe can attach to both primary probes, so that the analyte can be bound with greater affinity than when the 2° probe attaches to only one primary probe. In these embodiments, the analyte can be advantageously bound with greater affinity, even under more stringent conditions such as higher temperatures, lower salt, and/or presence of organic solvent.

FIG. 8A and FIG. 8B illustrate an embodiment 1000 for processing, isolating and/or enriching nucleic acid molecules. In FIG. 8A, a target analyte 1010 is illustrated, which may have a first target analyte sequence 1011 represented by “abcd” and a second target analyte sequence 1013 represented by “ijkl.” FIG. 8A shows a primary (1°) probe 1020, which may have a target analyte capture sequence 1021, represented by “a′b′c′d′.” The 1° probe 1020 may have a universal capture sequence 1022, represented by “efgh.” FIG. 8A shows an additional primary (1°) probe 1030, which may have a target analyte capture sequence 1031, represented by “i′j′k′l′.” The 1° probe 1030 may have a universal capture sequence 1034, represented by “mnop.” FIG. 8A shows a secondary (2°) probe 1060, which may have two or more 1° probe capture sequences represented by “h′g′f′e′” 1062 and “p′o′n′m′” 1064. The 2° probe 1060 may have an attached bait molecule 1069. FIG. 8B shows an operational embodiment having the target analyte 1010 bound to the 1° probes 1020 and 1030, which are each in turn bound to 2° probe 1060.

Referring to FIG. 8A and FIG. 8B, because the 2° probe attaches to both primary probes, in some embodiments, the analyte can be bound in a locked, multi-strand superstructure. The locked, multi-strand superstructure can be formed when the first and second target sequences 1011 and 1013 are in proximity to each other, the target analyte capture sequence 1021 is in proximity to the universal capture sequence 1022 of the same primary probe, the target analyte capture sequence 1031 is in proximity to the universal capture sequence 1034 of the same primary probe, and the 1° probe capture sequences 1062 and 1064 are in proximity to each other.

FIG. 8B shows an operational embodiment having the target analyte 1010 bound to the 1° probes 1020 and 1030, which are each in turn bound to 2° probe 1060. In these embodiments, the 2° probe can attach to both primary probes, so that the analyte can be bound with greater affinity than when the 2° probe attaches to only one primary probe. In these embodiments, the analyte can be advantageously bound with greater affinity, even under more stringent conditions such as higher temperatures, lower salt, and/or presence of organic solvent.

FIG. 9A and FIG. 9B illustrate an embodiment 1100 for processing, isolating and/or enriching nucleic acid molecules. In FIG. 9A, a target analyte 1110 is illustrated, which may have a first target analyte sequence 1111 represented by “abcd” and a second target analyte sequence 1113 represented by “ijkl,” and a third target analyte sequence 1115 represented by “qrst.” FIG. 9A shows a primary (1°) probe 1120, which may have a target analyte capture sequence 1121, represented by “a′b′c′d′.” The 1° probe 1120 may have a universal capture sequence 1122, represented by “efgh.” FIG. 9A shows an additional primary (1°) probe 1130, which may have a target analyte capture sequence 1133, represented by “i′j′k′l′.” The 1° probe 1130 may have a universal capture sequence 1134, represented by “mnop.” FIG. 9A shows an additional primary (1°) probe 1140, which may have a target analyte capture sequence 1145, represented by “q′r′s′t′.” The 1° probe 1140 may have a universal capture sequence 1146, represented by “uvwx.” FIG. 9A shows a secondary (2°) probe 1160, which may have three or more 1° probe capture sequences 1162 represented by “h′g′f′e′,” and 1164 represented by “p′o′n′m′,” and 1166 represented by “x′w′v′u′.” The 2° probe 1160 may have an attached bait molecule 1169. FIG. 9B shows an operational embodiment having the target analyte 1110 bound to the 1° probes 1120 and 1130 and 1140, which are each in turn bound to 2° probe 1160.

Referring to FIG. 9A and FIG. 9B, because the 2° probe attaches to a plurality of primary probes, in some embodiments, the analyte can be bound in a locked, multi-strand superstructure, as described above.

FIG. 9B shows an operational embodiment having the target analyte 1110 bound to the 1° probes 1120, 1130 and 1140, which are each in turn bound to 2° probe 1160. In these embodiments, the 2° probe can attach to three primary probes, so that the analyte can be bound with greater affinity than when the 2° probe attaches to only one or two primary probes. In these embodiments, the analyte can be advantageously bound with greater affinity, even under more stringent conditions such as higher temperatures, lower salt, and/or presence of organic solvent.

FIG. 10A and FIG. 10B illustrate an embodiment 1200 for processing, isolating and/or enriching nucleic acid molecules. In FIG. 10A, a target analyte 1210 is illustrated, which may have a first target analyte sequence 1211 represented by “abcd” and a second target analyte sequence 1213 represented by “ijkl,” and a third target analyte sequence 1215 represented by “qrst.” FIG. 10A shows a primary (1°) probe 1220, which may have a target analyte capture sequence 1221, represented by “a′b′c′d′.” The 1° probe 1220 may have a universal sequence 1222, represented by “efgh.” FIG. 10A shows an additional primary (1°) probe 1230, which may have a target analyte capture sequence 1233, represented by “i′j′k′l′.” The 1° probe 1230 may have a universal capture sequence 1234, represented by “mnop.” FIG. 10A shows an additional primary (1°) probe 1240, which may have a target analyte capture sequence 1245, represented by “q′r′s′t′.” The 1° probe 1240 may have a universal capture sequence 1246, represented by “uvwx.” FIG. 10A shows a secondary (2°) probe 1260, which may have three or more 1° probe capture sequences 1262 represented by “e′f′g′h′,” and 1264 represented by “m′n′o′p′,” and 1266 represented by “u′v′w′x′.” The 2° probe 1260 may have an attached bait molecule 1269. FIG. 10B shows an operational embodiment having the target analyte 1210 bound to the 1° probes 1220 and 1230 and 1240, which are each in turn bound to 2° probe 1260.

FIG. 10B shows an operational embodiment having the target analyte 1210 bound to the 1° probes 1220, 1230 and 1240, which are each in turn bound to 2° probe 1260. In these embodiments, the 2° probe can attach to three primary probes, so that the analyte can be bound with greater affinity than when the 2° probe attaches to only one or two primary probes. In these embodiments, the analyte can be advantageously bound with greater affinity, even under more stringent conditions such as higher temperatures, lower salt, and/or presence of organic solvent.

FIG. 11 shows an example of a microfluidic channel structure 1300 for partitioning individual biological particles.

FIG. 12 shows an example of a microfluidic channel structure 1400 for delivering barcode carrying beads to droplets.

FIG. 13 shows an example of a microfluidic channel structure for the controlled partitioning of beads into discrete droplets.

FIG. 14 shows an exemplary barcode carrying bead.

FIG. 15 shows an exemplary microwell array schematic.

FIG. 16 shows an exemplary workflow for processing nucleic acid molecules.

FIG. 17 describes exemplary labeling agents comprising reporter oligonucleotides attached thereto.

FIG. 18A schematically shows an example of labeling agents. FIG. 18B schematically shows another example workflow for processing nucleic acid molecules. FIG. 18C schematically shows another example workflow for processing nucleic acid molecules.

FIG. 19 is a schematic drawing illustrating an example of a spatial array oligonucleotide spatial capture probe.

### DETAILED DESCRIPTION OF THE DISCLOSURE

This invention provides methods, compositions, kits and systems for processing, isolating and/or enriching nucleic acid molecules. In some aspects, this disclosure provides methods for enriching nucleic acid molecules that can be used for transcriptomics. Methods of this invention can detect nucleic acid molecules of a sample that may be masked by highly expressed molecules.

Embodiments of this invention provide methods and compositions for processing, isolating or enriching nucleic acid molecules of interest. Nucleic acid molecules of interest can be detected with reduced DNA contamination.

In certain aspects, methods and compositions of this disclosure can be used to detect a wide variety of different samples containing nucleic acid molecules from different compartments. The methods and compositions of this invention can be used to reduce the presence of unprocessed or partially processed nucleic acid molecules and enhance the determination of molecules of interest.

In some embodiments, this invention provides methods and compositions for transcriptome sequencing with efficient capture molecules that specifically bind a large number of target regions of interest. In certain embodiments, the efficient capture molecules may have sequences that do not derive from any target of interest. The efficient capture molecules may reduce the amount of sequencing work needed to detect target regions of interest.

In further aspects, the methods, compositions and systems of this disclosure for processing, isolating and/or enriching nucleic acid molecules of interest can be used to broadly detect, sequence and represent target regions and transcript species with high specificity and efficiency.

As used herein, the term processing can refer to transforming the nature of a mixture of molecules. For example, a mixture of molecules can be transformed by changing the relative amounts of different molecules with the mixture. Processing a mixture can involve refining, purifying, filtering, purging, diluting, extracting, recovering, or recycling molecules of the mixture.

In additional embodiments, the methods, compositions and systems of this invention can be used for isolating target nucleic acid molecules for transcriptomics. This invention can provide efficient capture of target nucleic acid molecules of interest.

Embodiments of this invention can be used for enriching specific nucleic acid target molecules in complex populations of nucleic acid molecules. For example, specific nucleic acid target molecules can be enriched in complex populations of nucleic acid libraries.

In some embodiments, a target analyte nucleic acid molecule can be bound to a primary (1°) probe (also referred to herein as primary oligonucleotide or 1° oligonucleotide), which in turn can be bound to a secondary (2°) probe (also referred to herein as secondary oligonucleotide or 2° oligonucleotide). 1° probes can comprise a target binding sequence (also referred to herein as “target analyte capture sequence”), and a universal capture sequence. 2° probes can comprise a universal sequence (also referred to herein as 1° probe capture sequence or primary probe capture sequence) complementary to at least a portion of the universal capture sequence of one or more 1° probes.

A 2° probe may comprise one or more universal sequences, which may all be the same sequence. A 2° probe having a plurality of spaced-apart universal sequences, which are all the same sequence, may bind to a plurality of primary probes, each primary probe comprising the same cognate of the universal sequences of the 2° probe. This arrangement of primary and secondary probes advantageously provides the ability of a single 2° probe to be used to isolate or enrich a plurality of desired target molecules.

As used herein, a universal sequence is a sequence which can bind to its cognate on a primary probe. In some embodiments, all primary probes may contain the same universal capture sequence which is cognate to a universal sequence in a secondary probe, and therefore a single secondary probe can bind one or more desired target molecules via such primary probes. These embodiments can efficiently capture for isolation and/or enrichment all target analyte nucleic acid molecules, even in a library of molecules. A bait molecule can be attached to a 2° probe.

As used herein, the terms universal sequence and universal capture sequence can refer to a sequence that is contiguous or non-contiguous.

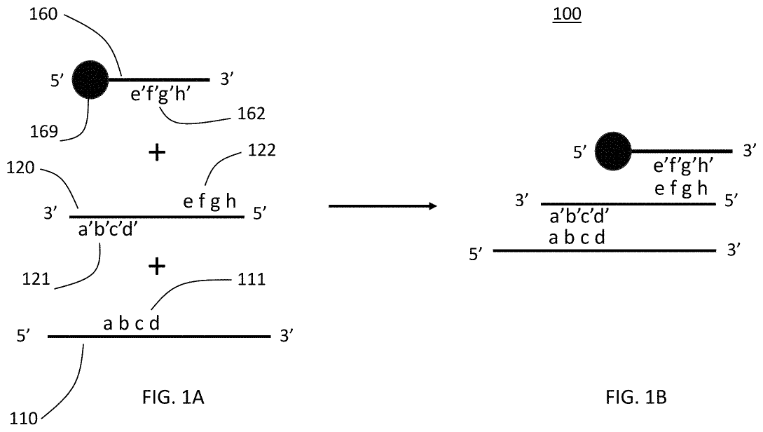

In operation, nucleic acid molecules can be isolated and/or enriched as shown in the system 100 of FIG. 1A. In FIG. 1A, a target analyte 110 is illustrated. The target analyte 110 may be a nucleic acid. In some examples, the target analyte 110 may be a cDNA. The target analyte 110 may have a nucleotide sequence, represented by “abcd” which may be called a target analyte sequence 111. Also illustrated in FIG. 1A is a primary (1°) probe 120. The 1° probe 120 may be a nucleic acid. The 1° probe 120 may have a target analyte capture sequence 121, represented by “a′b′c′d′.” The target analyte capture sequence 121 in the 1° probe 120 can be complementary to the target analyte sequence 111 in the target analyte 110. The target analyte capture sequence 121 and the target analyte sequence 111 can be capable of hybridizing to one another. The 1° probe 120 may have a universal capture sequence 122, represented by “efgh” in FIG. 1A. Also illustrated in FIG. 1A is a secondary (2°) probe 160. The 2° probe 160 may be a nucleic acid. The 2° probe 160 may have a universal sequence 162, here represented by “e′f′g′h′,” (which may also be referred to herein as a 1° probe capture sequence). The universal sequence 162 in the 2° probe 160 can be complementary to the universal capture sequence 122 in the 1° probe 120. The universal sequence 162 and the universal capture sequence 122 can be capable of hybridizing to one another. The 2° probe 160 may have an attached bait molecule 169 that has binding affinity to a binding partner. The bait molecule and its binding partner may be an affinity group. In some examples, the bait molecule 169 may be biotin. FIG. 1B shows an operational embodiment having the target analyte 110 hybridized to the 1° probe 120, which in turn is hybridized to the 2° probe 160.

As used herein, the terms target analyte sequence and target analyte capture sequence can refer to a sequence that is contiguous or non-contiguous.

As used herein and in the accompanying drawings, sequences designated as “abcd,” “a′b′c′d′,” “efgh,” and “e′f′g′h′,” as well as those with other letters representing nucleotides, are intended to represent nucleotide sequences of any number of nucleotides, i.e. any length. For example any of the sequences designated as “abcd,” “a′b′c′d′,” “efgh,” and “e′f′g′h′,” as well as those with other letters representing nucleotides, can be a sequence of two or more nucleotides, or three or more nucleotides, or four or more nucleotides, or five or more nucleotides, or one hundred or more nucleotides, and so forth, without limitation.

In further embodiments, a bait molecule can be attached to the 3′ end of the 2° probe.

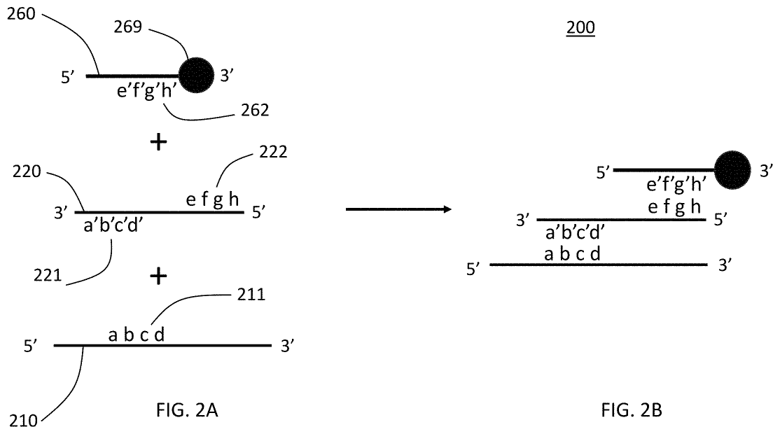

In operation, nucleic acid molecules can be isolated and/or enriched as shown in the system 200 of FIG. 2A. In FIG. 2A, a target analyte 210 is illustrated, which may have a target analyte sequence 211 represented by “abcd.” FIG. 2A shows a primary (1°) probe 220, which may have a target analyte capture sequence 221, represented by “a′b′c′d′.” The 1° probe 220 may have a universal capture sequence 222, represented by “efgh.” FIG. 2A shows a secondary (2°) probe 260, which may have a 1° probe capture sequence 262 represented by “e′f′g′h′.” The 2° probe 260 may have an attached bait molecule 269. FIG. 2B shows an operational embodiment having the target analyte 210 bound to the 1° probe 220, which in turn is bound to the 2° probe 260.

In additional embodiments, a target analyte nucleic acid molecule can be captured bound to a primary (1°) probe, which in turn can be bound to a secondary (2°) probe. In these embodiments, the primary probe can contain one region to bind a cognate on a target which is spaced apart from a second region that binds to a secondary probe universal sequence. These embodiments can efficiently capture for isolation and/or enrichment all target analyte nucleic acid molecules, even in a library of molecules. In additional embodiments, a bait molecule can be attached to the 5′ end of the 2° probe.

In certain embodiments, a target analyte nucleic acid molecule can bind to two or more primary (1°) probes, which in turn can each be bound to a secondary (2°) probe. In these embodiments, the primary probes can contain one region to bind a cognate on a particular target and a second region that binds to a secondary probe universal sequence. The primary probes can bind to the target analyte with proximity to each other. These embodiments can efficiently capture for isolation and/or enrichment all target analyte nucleic acid molecules, even in a library of molecules. A bait molecule can be attached to the 5′ end or 3′ end of the secondary probe. In these embodiments, the analyte can be advantageously bound with even greater affinity, and under more stringent conditions such as higher temperatures, lower salt, and/or presence of organic solvent.

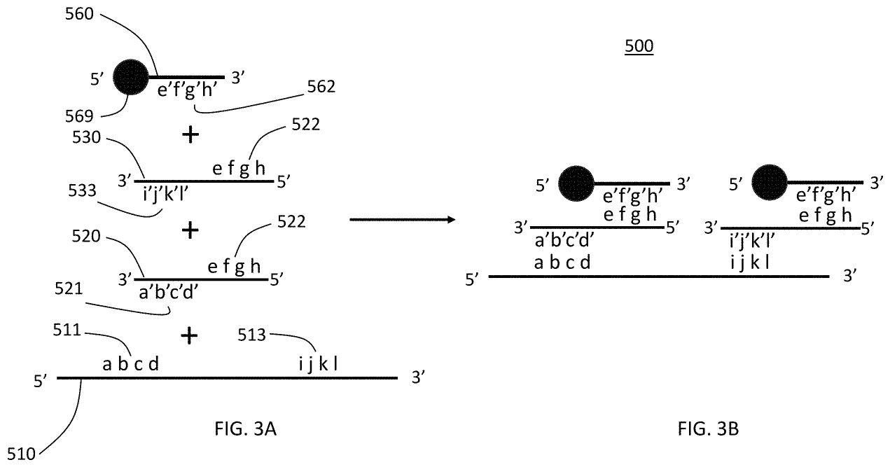

In operation, nucleic acid molecules can be isolated and/or enriched as shown in the system 500 of FIG. 3A. In FIG. 3A, a target analyte 510 is illustrated, which may have a first target analyte sequence 511 represented by “abcd” and a second target analyte sequence 513 represented by “ijkl.” FIG. 3A shows a primary (1°) probe 520, which may have a target analyte capture sequence 521, represented by “a′b′c′d′.” The 1° probe 520 may have a universal capture sequence 522, represented by “efgh.” FIG. 3A shows an additional primary (1°) probe 530, which may have a target analyte capture sequence 533, represented by “i′j′k′l′.” The 1° probe 530 may have a universal capture sequence 522, represented by “efgh.” The universal capture sequence 522 of 1° probe 520 and 1° probe 530 may be the same sequence. FIG. 3A shows a secondary (2°) probe 560, which may have a 1° probe capture sequence 562 represented by “e′f′g′h′.” The 2° probe 560 may have an attached bait molecule 569. FIG. 3B shows an operational embodiment having the target analyte 510 bound to the 1° probes 520 and 530, which in turn are bound to 2° probes 560.

In further embodiments, a target analyte nucleic acid molecule can bind to two or more primary (1°) probes in different regions of the target analyte. The primary probes can in turn each be bound to a different secondary (2°) probe. In these embodiments, the primary probes can contain one region to bind a cognate on a target and a second region that binds to a secondary probe universal sequence, where each primary probe binds to a different secondary probe with a different universal sequence. The primary probes bind to the target analyte with proximity to each other. For example, the primary probes can bind to adjacent sequences of the target analyte, or non-contiguous sequences spaced no more than, e.g., 10 nucleotides apart, 5 nucleotides apart, 4 nucleotides apart, 3 nucleotides apart, 2 nucleotides apart, or 1 nucleotide apart. These embodiments can efficiently capture for isolation and/or enrichment all target analyte nucleic acid molecules, even in a library of molecules. A bait molecule can be attached to the 5′ end or 3′ end of the 2° probe. In these embodiments, the analyte can be advantageously bound with even greater affinity, and under more stringent conditions such as higher temperatures, lower salt, and/or presence of organic solvent.

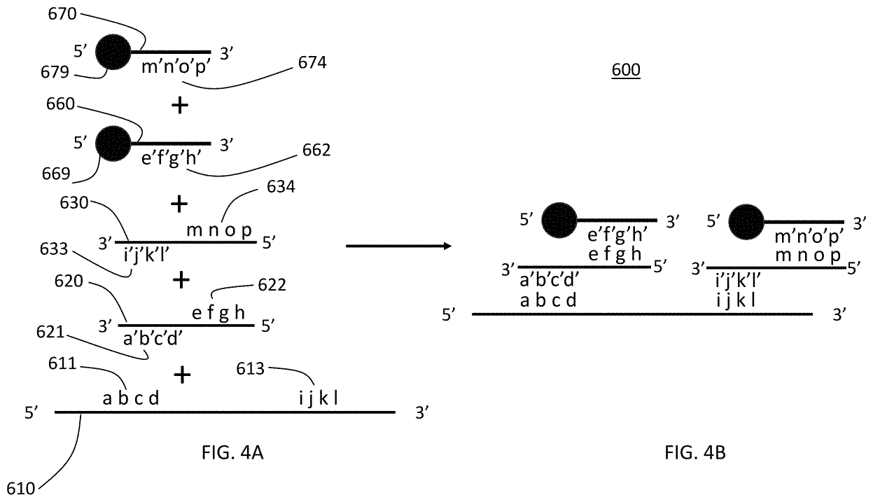

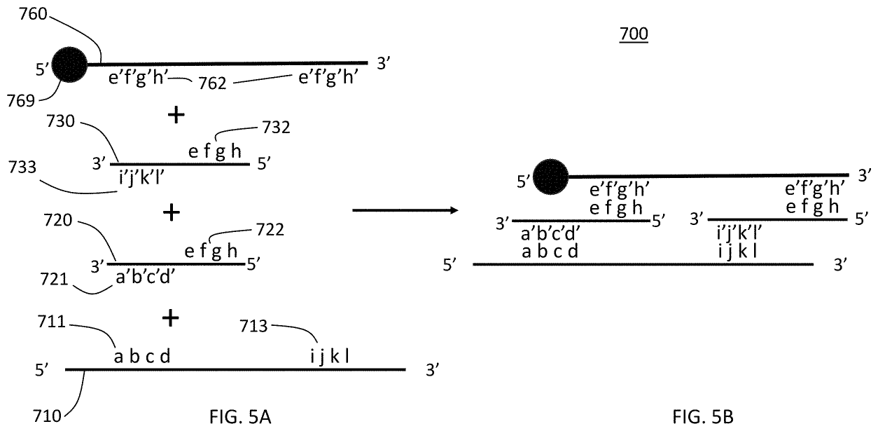

In operation, nucleic acid molecules can be isolated and/or enriched as shown in the system 600 of FIG. 4A. In FIG. 4A, a target analyte 610 is illustrated, which may have a first target analyte sequence 611 represented by “abcd” and a second target analyte sequence 613 represented by “ijkl.” FIG. 4A shows a primary (1°) probe 620, which may have a target analyte capture sequence 621, represented by “a′b′c′d′.” The 1° probe 620 may have a universal capture sequence 622, represented by “efgh.” FIG. 4A shows an additional primary (1°) probe 630, which may have a target analyte capture sequence 633, represented by “i′j′k′l′.” The 1° probe 630 may have a universal capture sequence 634, represented by “mnop.” The universal capture sequence 622 of primary (1°) probe 620 and the universal capture sequence 634 of 1° probe 630 may be different sequences. FIG. 4A shows a secondary (2°) probe 660, which may have a 1° probe capture sequence 662 represented by “e′f′g′h′.” The 2° probe 660 may have an attached bait molecule 669. FIG. 4A shows a secondary (2°) probe 670, which may have a 1° probe capture sequence 674 represented by “m′n′o′p′.” The 2° probe 670 may have an attached bait molecule 679. FIG. 4B shows an operational embodiment having the target analyte 610 bound to the 1° probes 620 and 630, which are each in turn bound to 2° probes 660 and 670.

In further embodiments, a target analyte nucleic acid molecule can bind to two or more primary (1°) probes in different regions of the target analyte. The primary probes can in turn each be bound to a single secondary (2°) probe, where the secondary probes have different universal sequences. In these embodiments, the primary probes can contain one region to bind a cognate on a target and a second region that binds to a universal sequence of a secondary probe, where each primary probe binds to a different secondary probe with a different universal sequence. A bait molecule can be attached to each secondary probe.

In some embodiments, the primary probes can bind to the target analyte with proximity to each other. In these embodiment, the target analytes can be efficiently captured for isolation and/or enrichment from a mixture or library of molecules. In these embodiments, the analyte can be advantageously bound with even greater affinity, and under more stringent conditions such as higher temperatures, lower salt, and/or presence of organic solvent.

In operation, nucleic acid molecules can be isolated and/or enriched as shown in the system 700 of FIG. 5A. In FIG. 5A, a target analyte 710 is illustrated, which may have a first target analyte sequence 711 represented by “abcd” and a second target analyte sequence 713 represented by “ijkl.” FIG. 5A shows a primary (1°) probe 720, which may have a target analyte capture sequence 721, represented by “a′b′c′d′.” The 1° probe 720 may have a universal capture sequence 722, represented by “efgh.” FIG. 5A shows an additional primary (1°) probe 730, which may have a target analyte capture sequence 733, represented by “i′j′k′l′.” The 1° probe 730 may have a universal capture sequence 732, represented by “efgh.” FIG. 5A shows a secondary (2°) probe 760, which may have two or more 1° probe capture sequences 762 represented by “e′f′g′h′.” The 2° probe 760 may have an attached bait molecule 769. FIG. 5B shows an operational embodiment having the target analyte 710 bound to the 1° probes 720 and 730, which are each in turn bound to 2° probe 760.

FIG. 5B shows an operational embodiment having the target analyte 710 bound to the 1° probes 720 and 730, which are each in turn bound to 2° probe 760. In these embodiments, the 2° probe can attach to both primary probes, so that the analyte can be bound with greater affinity than when the 2° probe attaches to only one primary probe. In these embodiments, the analyte can be advantageously bound with greater affinity, even under more stringent conditions such as higher temperatures, lower salt, and/or presence of organic solvent. In additional embodiments, a target analyte may bind to a plurality of 1° probes, which are each in turn bound to a single 2° probe. In these additional embodiments, the analyte can be advantageously bound with even greater affinity, and under more stringent conditions such as higher temperatures, lower salt, and/or presence of organic solvent.

In certain embodiments, a target analyte nucleic acid molecule can bind to two or more primary (1°) probes in different regions of the target analyte. The primary probes can in turn each be bound to the same, single secondary (2°) probe. In these embodiments, the primary probes can contain one region to bind a cognate on a target and a second region that binds to a secondary probe universal sequence, where each primary probe binds to a different region of the same secondary probe with a different universal sequence. The primary probes can bind to the target analyte with proximity to each other. These embodiments can efficiently capture for isolation and/or enrichment all target analyte nucleic acid molecules, even in a library of molecules. A bait molecule can be attached to the 5′ end or 3′ end of the secondary probe. In these embodiments, the analyte can be advantageously bound with even greater affinity, and under more stringent conditions such as higher temperatures, lower salt, and/or presence of organic solvent.

In operation, nucleic acid molecules can be isolated and/or enriched as shown in the system 800 of FIG. 6A. In FIG. 6A, a target analyte 810 is illustrated, which may have a first target analyte sequence 811 represented by “abcd” and a second target analyte sequence 813 represented by “ijkl.” FIG. 6A shows a primary (1°) probe 820, which may have a target analyte capture sequence 821, represented by “a′b′c′d′.” The 1° probe 820 may have a universal capture sequence 822, represented by “efgh.” FIG. 6A shows an additional primary (1°) probe 830, which may have a target analyte capture sequence 833, represented by “i′j′k′l′.” The 1° probe 830 may have a universal capture sequence 834, represented by “mnop.” The universal capture sequence 822 and universal capture sequence 834 may be different sequences. FIG. 6A shows a secondary (2°) probe 860, which may have two or more 1° probe capture sequences 862 represented by “e′f′g′h′,” and 864 represented by “m′n′o′p′.” The 2° probe 860 may have an attached bait molecule 869. FIG. 6B shows an operational embodiment having the target analyte 810 bound to the 1° probes 820 and 830, which are each in turn bound to 2° probe 860.

FIG. 6B shows an operational embodiment having the target analyte 810 bound to the 1° probes 820 and 830, which are each in turn bound to 2° probe 860. In these embodiments, the 2° probe can attach to both primary probes, so that the analyte can be bound with greater affinity than when the 2° probe attaches to only one primary probe. In these embodiments, the analyte can be advantageously bound with greater affinity, even under more stringent conditions such as higher temperatures, lower salt, and/or presence of organic solvent. In additional embodiments, a target analyte may bind to a plurality of 1° probes, which are each in turn bound to a single 2° probe. In these additional embodiments, the analyte can be advantageously bound with even greater affinity, and under more stringent conditions such as higher temperatures, lower salt, and/or presence of organic solvent.

In certain embodiments, a target analyte nucleic acid molecule can bind to two or more primary (1°) probes in different regions of the target analyte. The primary probes can in turn each be bound to the same, single secondary (2°) probe. In these embodiments, the primary probes can contain one region to bind a cognate on a target and a spaced apart second region that binds to a secondary probe universal sequence, where each primary probe binds to the same, but spaced apart cognate region of the same secondary probe with the same universal sequence. The primary probes can bind to the target analyte with proximity to each other. These embodiments can efficiently capture for isolation and/or enrichment all target analyte nucleic acid molecules, even in a library of molecules. A bait molecule can be attached to the 5′ end or 3′ end of the secondary probe. In these embodiments, the analyte can be advantageously bound with even greater affinity, and under more stringent conditions such as higher temperatures, lower salt, and/or presence of organic solvent.

In operation, nucleic acid molecules can be isolated and/or enriched as shown in the system 900 of FIG. 7A. In FIG. 7A, a target analyte 910 is illustrated, which may have a first target analyte sequence 911 represented by “abcd” and a second target analyte sequence 913 represented by “ijkl.” FIG. 7A shows a primary (1°) probe 920, which may have a target analyte capture sequence 921, represented by “a′b′c′d′.” The 1° probe 920 may have a universal capture sequence 922, represented by “efgh.” FIG. 7A shows an additional primary (1°) probe 930, which may have a target analyte capture sequence 933, represented by “i′j′k′l′.” The 1° probe 930 may have a universal capture sequence 922, represented by “efgh.” FIG. 7A shows a secondary (2°) probe 960, which may have two or more 1° probe capture sequences 962 each represented by “h′g′f′e′.” The 2° probe 960 may have an attached bait molecule 969. FIG. 7B shows an operational embodiment having the target analyte 910 bound to the 1° probes 920 and 930, which are each in turn bound to 2° probe 960.

FIG. 7B shows an operational embodiment having the target analyte 910 bound to the 1° probes 920 and 930, which are each in turn bound to 2° probe 960. In these embodiments, the 2° probe can attach to both primary probes, so that the analyte can be bound with greater affinity than when the 2° probe attaches to only one primary probe. In these embodiments, the analyte can be advantageously bound with greater affinity, even under more stringent conditions such as higher temperatures, lower salt, and/or presence of organic solvent. In additional embodiments, a target analyte may bind to a plurality of 1° probes, which are each in turn bound to a single 2° probe. In these additional embodiments, the analyte can be advantageously bound with even greater affinity, and under more stringent conditions such as higher temperatures, lower salt, and/or presence of organic solvent.

In further embodiments, a target analyte nucleic acid molecule can bind to two or more primary (1°) probes in different regions of the target analyte. The primary probes can in turn each be bound to the same, single secondary (2°) probe. In these embodiments, the primary probes can contain one region to bind a cognate on a target and a spaced apart second region that binds to a secondary probe universal sequence, where each primary probe binds to a different, but spaced apart cognate region of the same secondary probe with a different universal sequence. The primary probes can bind to the target analyte with proximity to each other. These embodiments can efficiently capture for isolation and/or enrichment all target analyte nucleic acid molecules, even in a library of molecules. A bait molecule can be attached to the 5′ end or 3′ end of the secondary probe. In these embodiments, the analyte can be advantageously bound with even greater affinity, and under more stringent conditions such as higher temperatures, lower salt, and/or presence of organic solvent.

In operation, nucleic acid molecules can be isolated and/or enriched as shown in the system 1000 of FIG. 8A. In FIG. 8A, a target analyte 1010 is illustrated, which may have a first target analyte sequence 1011 represented by “abcd” and a second target analyte sequence 1013 represented by “ijkl.” FIG. 8A shows a primary (1°) probe 1020, which may have a target analyte capture sequence 1021, represented by “a′b′c′d′.” The 1° probe 1020 may have a universal capture sequence 1022, represented by “efgh.” FIG. 8A shows an additional primary (1°) probe 1030, which may have a target analyte capture sequence 1031, represented by “i′j′k′l′.” The 1° probe 1030 may have a universal capture sequence 1034, represented by “mnop.” FIG. 8A shows a secondary (2°) probe 1060, which may have two or more 1° probe capture sequences 1062 represented by “h′g′f′e′,” and 1064 represented by “p′o′n′m′.” The 2° probe 1060 may have an attached bait molecule 1069. FIG. 8B shows an operational embodiment having the target analyte 1010 bound to the 1° probes 1020 and 1030, which are each in turn bound to 2° probe 1060.

FIG. 8B shows an operational embodiment having the target analyte 1010 bound to the 1° probes 1020 and 1030, which are each in turn bound to 2° probe 1060. In these embodiments, the 2° probe can attach to both primary probes, so that the analyte can be bound with greater affinity than when the 2° probe attaches to only one primary probe. In these embodiments, the analyte can be advantageously bound with greater affinity, even under more stringent conditions such as higher temperatures, lower salt, and/or presence of organic solvent. In additional embodiments, a target analyte may bind to a plurality of 1° probes, which are each in turn bound to a single 2° probe. In these additional embodiments, the analyte can be advantageously bound with even greater affinity, and under more stringent conditions such as higher temperatures, lower salt, and/or presence of organic solvent.

In additional embodiments, a target analyte nucleic acid molecule can bind to three or more primary (1°) probes in different regions of the target analyte. The primary probes can in turn each be bound to the same, single secondary (2°) probe. In these embodiments, the primary probes can contain one region to bind a cognate on a target and a spaced apart second region that binds to a secondary probe universal sequence, where each primary probe binds to a different, but spaced apart cognate region of the same secondary probe with a different universal sequence. The primary probes can bind to the target analyte with proximity to each other, either two at a time in proximity or three or more in proximity. These embodiments can efficiently capture for isolation and/or enrichment all target analyte nucleic acid molecules, even in a library of molecules. A bait molecule can be attached to the 5′ end or 3′ end of the secondary probe. In these embodiments, the analyte can be advantageously bound with even greater affinity, and under more stringent conditions such as higher temperatures, lower salt, and/or presence of organic solvent.

In operation, nucleic acid molecules can be isolated and/or enriched as shown in the system 1100 of FIG. 9A. In FIG. 9A, a target analyte 1110 is illustrated, which may have a first target analyte sequence 1111 represented by “abcd” and a second target analyte sequence 1113 represented by “ijkl,” and a third target analyte sequence 1115 represented by “qrst.” FIG. 9A shows a primary (1°) probe 1120, which may have a target analyte capture sequence 1121, represented by “a′b′c′d′.” The 1° probe 1120 may have a universal capture sequence 1122, represented by “efgh.” FIG. 9A shows an additional primary (1°) probe 1130, which may have a target analyte capture sequence 1133, represented by “i′j′k′l′.” The 1° probe 1130 may have a universal capture sequence 1134, represented by “mnop.” FIG. 9A shows an additional primary (1°) probe 1140, which may have a target analyte capture sequence 1145, represented by “q′r′s′t′.” The 1° probe 1140 may have a universal capture sequence 1146, represented by “uvwx.” FIG. 9A shows a secondary (2°) probe 1160, which may have three or more 1° probe capture sequences 1162 represented by “h′g′f′e′,” and 1164 represented by “p′o′n′m′,” and 1166 represented by “x′w′v′u′.” The 2° probe 1160 may have an attached bait molecule 1169. FIG. 9B shows an operational embodiment having the target analyte 1110 bound to the 1° probes 1120 and 1130 and 1140, which are each in turn bound to 2° probe 1160.

FIG. 9B shows an operational embodiment having the target analyte 1110 bound to the 1° probes 1120, 1130 and 1140, which are each in turn bound to 2° probe 1160. In these embodiments, the 2° probe can attach to three primary probes, so that the analyte can be bound with greater affinity than when the 2° probe attaches to only one or two primary probes. In these embodiments, the analyte can be advantageously bound with greater affinity, even under more stringent conditions such as higher temperatures, lower salt, and/or presence of organic solvent. In additional embodiments, a target analyte may bind to a plurality of 1° probes, which are each in turn bound to a single 2° probe. In these additional embodiments, the analyte can be advantageously bound with even greater affinity, and under more stringent conditions such as higher temperatures, lower salt, and/or presence of organic solvent.

In additional embodiments, a target analyte nucleic acid molecule can bind to three or more primary (1°) probes in different regions of the target analyte. The primary probes can in turn each be bound to the same, single secondary (2°) probe. In these embodiments, the primary probes can contain one region to bind a cognate on a target and a spaced apart second region that binds to a secondary probe universal sequence, where each primary probe binds to a different, but spaced apart cognate region of the same secondary probe with a different universal sequence. The primary probes can bind to the target analyte with proximity to each other, either two at a time in proximity or three or more in proximity. These embodiments can efficiently capture for isolation and/or enrichment all target analyte nucleic acid molecules, even in a library of molecules. A bait molecule can be attached to the 5′ end or 3′ end of the secondary probe. In these embodiments, the analyte can be advantageously bound with even greater affinity, and under more stringent conditions such as higher temperatures, lower salt, and/or presence of organic solvent.

In operation, nucleic acid molecules can be isolated and/or enriched as shown in the system 1200 of FIG. 10A. In FIG. 10A, a target analyte 1210 is illustrated, which may have a first target analyte sequence 1211 represented by “abcd” and a second target analyte sequence 1213 represented by “ijkl,” and a third target analyte sequence 1215 represented by “qrst.” FIG. 10A shows a primary (1°) probe 1220, which may have a target analyte capture sequence 1221, represented by “a′b′c′d′.” The 1° probe 1220 may have a universal capture sequence 1222, represented by “efgh.” FIG. 10A shows an additional primary (1°) probe 1230, which may have a target analyte capture sequence 1233, represented by “i′j′k′l′.” The 1° probe 1230 may have a universal capture sequence 1234, represented by “mnop.” FIG. 10A shows an additional primary (1°) probe 1240, which may have a target analyte capture sequence 1245, represented by “q′r′s′t′.” The 1° probe 1240 may have a universal capture sequence 1246, represented by “uvwx.” FIG. 10A shows a secondary (2°) probe 1260, which may have three or more 1° probe capture sequences 1262 represented by “e′f′g′h′,” and 1264 represented by “m′n′o′p′,” and 1266 represented by “u′v′w′x′.” The 2° probe 1260 may have an attached bait molecule 1269. FIG. 10B shows an operational embodiment having the target analyte 1210 bound to the 1° probes 1220 and 1230 and 1240, which are each in turn bound to 2° probe 1260.

FIG. 10B shows an operational embodiment having the target analyte 1210 bound to the 1° probes 1220, 1230 and 1240, which are each in turn bound to 2° probe 1260. In these embodiments, the 2° probe can attach to three primary probes, so that the analyte can be bound with greater affinity than when the 2° probe attaches to only one or two primary probes. In these embodiments, the analyte can be advantageously bound with greater affinity, even under more stringent conditions such as higher temperatures, lower salt, and/or presence of organic solvent. In additional embodiments, a target analyte may bind to a plurality of 1° probes, which are each in turn bound to a single 2° probe. In these additional embodiments, the analyte can be advantageously bound with even greater affinity, and under more stringent conditions such as higher temperatures, lower salt, and/or presence of organic solvent.

Embodiments of this invention provide methods for processing, isolating and/or enriching nucleic acid molecules without inducing significant alterations in the molecules or transcriptome being studied.

In some embodiments, primary probes may work together by binding to the target analyte with proximity to each other. The proximity can be very close. In some embodiments, primary probes may bind to the target analyte with proximity as being adjacent to each other. In certain embodiments, primary probes may bind to the target analyte with proximity as being nearly adjacent to each other. The separation between primary probes binding the target with proximity to each other can be from zero (adjacent nt) to four nucleotides.

The length of a primary probe cognate for binding to a target analyte can be from four to twenty nucleotides.

The length of a secondary probe cognate for binding to a primary probe can be from four to twenty nucleotides.

In general, hybridization of a nucleotide sequence present in a probe nucleic acid to a complementary nucleotide sequence present in a target nucleic acid can be used for detecting specific target molecules in complex populations of nucleic acid molecules, e.g., libraries.

In some embodiments, a probe nucleic acid may be used having an attached molecule, e.g., biotin, that can be specifically bound by a second molecule, e.g., streptavidin, and may be used to retrieve a target nucleic acid that is specifically hybridized to the probe, i.e., hybrid capture.

In further embodiments, a hybrid capture method may be used to probe and retrieve specific sequences from libraries of genes or other analytes, e.g., single cell or single nuclei libraries, including libraries that contain information on the spatial locations in tissues from which the analytes originated, e.g., as prepared using spatial methodologies described herein.

In some embodiments, a modular enrichment kit can be provided designed to enrich one or more target molecules of interest within a library, while decreasing sequencing requirements by up to 90%. Target enrichment can be achieved by a hybrid capture workflow. Target-specific 1° probes may be hybridized to their complement in the library. 2° probes may be hybridized to the target-specific bound 1° probes as described herein. The 2° probes may comprise a bait molecule, e.g., biotin, which can bind to a streptavidin bead, and then washed to remove non-targeted library molecules. The bead-bound, targeted library portion can be amplified to produce a sequencing-ready library. In some embodiments, a hybrid capture workflow may be performed with a panel of baits, which can be single stranded oligonucleotides with a 5′ biotin modification. Each bait can target a unique library molecule. For example, in a step of library capture, baits may be added to the concentrated library for hybridization, denaturation and hybridization, followed by the addition of streptavidin magnetic beads. The mix can be incubated to conjugate biotinylated baits to streptavidin beads. Subsequent washes may remove non-hybridized library molecules. Hybridized library molecules bound to streptavidin beads can be isolated using a magnetic field to attract the beads. The isolated library molecules may then be amplified and DNA sequences determined.

In some aspects, embodiments of this invention can provide enhanced spatial resolution in a spatial single cell analysis system for spatial transcriptomics and/or spatial gene expression. This invention contemplates enhanced single cell resolution capabilities. Measurements for determining the resolution of a single cell spatial system may include the ability to measure and determine the accuracy of cell segmentation, and the ability to measure and determine the distance of RNA diffusion. These factors can contribute to enhanced resolution.

In further aspects, this invention can provide a cell-based system using different cell types which can be mixed or unmixed in defined proportions and applied to spatial transcriptomic analysis systems. The cells, having different populations being differentially fluorescently labeled, can be imaged and the data analyzed for cell segmentation measurements based on their different fluorescent signals. Additionally, nucleic acids captured from the cells can be sequenced and the data used to further segment cells based on the spatial barcodes associated with the captured analytes. Once the imaging and sequencing data are integrated and evaluated, diffusion of target analyte capture can also be determined. This invention further contemplates determining the resolution of a spatial transcriptomic system by way of cell segmentation and diffusion data analysis.

In alternative embodiments of the above methods, a biological sample may be probed for expression of specific proteins using antibodies. The antibodies may have attached oligonucleotide tags having a specific nucleotide sequence that can couple with a nucleic acid barcode molecule attached to a support, e.g., by hybridization.

In additional embodiments of the above methods, a biological sample may be probed for presence or absence of genetic mutations, variants, diversity, and/or polymorphisms in genomes, including single-nucleotide polymorphisms (SNPs) or single-nucleotide variants (SNVs) in genomes of cells making up the tissue. In some examples, a probe for a SNP or SNV may include a specific nucleotide sequence that can differentially hybridize to a genomic sequence dependent on whether a SNP or SNV is present. In further examples, a probe for a SNP or SNV may include a nucleotide sequence that can hybridize to a genomic sequence that is linked to, e.g., upstream of downstream of, a genome region that might contain the SNP or SNV. Extension of the hybridized sequence, using the region of the genome that might contain the SNP/SNV as a template, and nucleotide sequencing of the extension product, may be used to determine if the SNP/SNV is present in the extension product. In some examples, probes for specific SNPS or SNVs may be part of the capture domain of certain oligonucleotides that make up the oligonucleotide array. Other techniques may be used to detect SNPs and/or SNVs.

In further embodiments of the above methods, a biological sample may be probed for isoforms of genes, transcripts, e.g., alternative transcription start sites, alternatively-spliced mRNAs, or proteins. In some examples, a probe for an isoform of a gene or transcript may be designed to hybridize to one form but not the other, or may be designed to hybridize to or near a region that may contain the isoform such that amplification and/or extension of the hybridized probe, and optional nucleotide sequencing of the amplified product, can detect presence or absence of specific isoforms. In some examples, a probe for an isoform of a protein may be an antibody designed to differentially bind to the different isoforms. The antibodies used may have attached nucleotide tags that can capture domains of the barcoded molecules on a support, as described above.

Affinity groups of this disclosure include the streptavidin-biotin system, which is a protein-ligand interaction present in nature that has been successfully used in a number of applications including detection of proteins, nucleic acids and lipids as well as protein purification.

Additional examples of affinity groups of this disclosure include Glutathione and Glutathione S-transferase, Maltose and Maltose-binding Protein, as well as Chitin and Chitin-binding Protein.

Additional examples of affinity groups of this disclosure include a SPYCATCHER and SPYTAG system, as well as orthogonal systems thereof.

A capture domain of a probe can contain a region of complementarity. The capture domain of a primary probe may contain a region of complementarity to a nucleic acid of interest, e.g. a RNA or an mRNA.

A capture domain of this disclosure may include six or more nucleotides, or 10 or more nucleotides, or 15 or more nucleotides, or 20 or more nucleotides, or 25 or more nucleotides, or 30 or more nucleotides, or 35 or more nucleotides. A capture domain of this disclosure may contain 6-10 nucleotides, or 6-35 nucleotides, or 10-14 nucleotides, or 10-15 nucleotides, or 10-20 nucleotides, or 10-30 nucleotides, or 10-35 nucleotides, or 10-50 nucleotides, or 15-25 nucleotides, or 15-30 nucleotides, or 15-35 nucleotides, or 20-50 nucleotides, or 25-35 nucleotides.

A poly-T element of a capture domain of this disclosure may include six or more nucleotides, or 10 or more nucleotides, or 15 or more nucleotides, or 20 or more nucleotides, or 30 or more nucleotides, or 35 or more nucleotides. A poly-T element of a capture domain of this disclosure may contain 6-10 nucleotides, or 6-35 nucleotides, or 10-14 nucleotides, or 10-15 nucleotides, or 10-20 nucleotides, or 10-30 nucleotides, or 10-35 nucleotides, or 10-50 nucleotides, or 15-25 nucleotides, or 15-30 nucleotides, or 15-35 nucleotides, or 20-50 nucleotides, or 25-35 nucleotides.

A capture domain of this disclosure may have about 11 nucleotides. A capture domain of this disclosure may have about 12 nucleotides. A capture domain of this disclosure may have about 13 nucleotides. A capture domain of this disclosure may have about 14 nucleotides. A capture domain of this disclosure may have about 15 nucleotides. A capture domain of this disclosure may have about 16 nucleotides. A capture domain of this disclosure may have about 17 nucleotides. A capture domain of this disclosure may have about 18 nucleotides. A capture domain of this disclosure may have about 19 nucleotides.

A poly-T element of a capture domain of this disclosure may have about 11 nucleotides. A poly-T element of a capture domain of this disclosure may have about 12 nucleotides. A poly-T element of a capture domain of this disclosure may have about 13 nucleotides. A poly-T element of a capture domain of this disclosure may have about 14 nucleotides. A poly-T element of a capture domain of this disclosure may have about 15 nucleotides. A poly-T element of a capture domain of this disclosure may have about 16 nucleotides. A poly-T element of a capture domain of this disclosure may have about 17 nucleotides. A poly-T element of a capture domain of this disclosure may have about 18 nucleotides. A poly-T element of a capture domain of this disclosure may have about 19 nucleotides.

A capture domain of this disclosure may have 11 nucleotides, or 12 nucleotides, or 13 nucleotides, or 14 nucleotides, or 15 nucleotides, or 16 nucleotides, or 17 nucleotides, or 18 nucleotides, or 19 nucleotides.

A poly-T element of a capture domain of this disclosure may have 11 nucleotides, or 12 nucleotides, or 13 nucleotides, or 14 nucleotides, or 15 nucleotides, or 16 nucleotides, or 17 nucleotides, or 18 nucleotides, or 19 nucleotides.

In some cases, the target nucleic acid molecule is from a nucleic acid library prepared using a partitioning method disclosed herein. In some aspects, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. A partition can be a volume or subvolume wherein diffusion of contents beyond the volume or sub-volume is inhibited. For example, the partitions can include a porous matrix that is capable of entraining and/or retaining materials within its matrix. The partition can be a droplet in an emulsion or a well. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead.

Unique identifiers, such as barcodes, may be injected into the droplets previous to, subsequent to, or concurrently with droplet generation, such as via a microcapsule (e.g., bead), as described elsewhere herein.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

The partitions described herein may comprise small volumes, for example, less than about 10 microliters (μL), 5 μL, 1 μL, 10 nanoliters (nL), 5 nL, 1 nL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

Partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Application No. PCT/US20/17785, which is entirely incorporated herein by reference for all purposes.

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

FIG. 11 shows an example of a microfluidic channel structure 1300 for partitioning individual biological particles. The channel structure 1300 can include channel segments 1302, 1304, 1306 and 1308 communicating at a channel junction 1310. In operation, a first aqueous fluid 1312 that includes suspended biological particles (or cells) 1314 may be transported along channel segment 1302 into junction 1310, while a second fluid 1316 that is immiscible with the aqueous fluid 1312 is delivered to the junction 1310 from each of channel segments 1304 and 1306 to create discrete droplets 1318, 1320 of the first aqueous fluid 1312 flowing into channel segment 1308, and flowing away from junction 1310. The channel segment 1308 may be fluidically coupled to an outlet reservoir where the discrete droplets can be stored and/or harvested. A discrete droplet generated may include an individual biological particle 1314 (such as droplets 1318). A discrete droplet generated may include more than one individual biological particle 1314 (not shown in FIG. 11). A discrete droplet may contain no biological particle 1314 (such as droplet 1320). Each discrete partition may maintain separation of its own contents (e.g., individual biological particle 1314) from the contents of other partitions.

Referring to FIG. 11, the second fluid 1316 can comprise an oil, such as a fluorinated oil, that includes a fluorosurfactant for stabilizing the resulting droplets, for example, inhibiting subsequent coalescence of the resulting droplets 1318, 1320. Examples of particularly useful partitioning fluids and fluorosurfactants are described, for example, in U.S. Patent Application Publication No. 2010/0105112, which is entirely incorporated herein by reference for all purposes.

Referring to FIG. 11, as will be appreciated, the channel segments described herein may be coupled to any of a variety of different fluid sources or receiving components, including reservoirs, tubing, manifolds, or fluidic components of other systems. As will be appreciated, the microfluidic channel structure 1300 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Referring to FIG. 11, the generated droplets may comprise two subsets of droplets: (1) occupied droplets 1318, containing one or more biological particles 1314, and (2) unoccupied droplets 1320, not containing any biological particles 1314. Occupied droplets 1318 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

Referring to FIG. 11, in some cases, it may be desirable to minimize the creation of excessive numbers of empty partitions, such as to reduce costs and/or increase efficiency. While this minimization may be achieved by providing a sufficient number of biological particles (e.g., biological particles 1314) at the partitioning junction 1310, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above.

As will be appreciated, the above-described occupancy rates are also applicable to partitions that include both biological particles and additional reagents, such as microcapsules or beads (e.g., gel beads) carrying barcoded nucleic acid molecules (e.g., oligonucleotides).

In some examples, a partition of the plurality of partitions may comprise a single biological particle or biological particle (e.g., a single cell or a single nucleus of a cell). In some examples, a partition of the plurality of partitions may comprise multiple biological particles. Such partitions may be referred to as multiply occupied partitions, and may comprise, for example, two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

Microfluidic systems for partitioning are further described in U.S. Patent Application Pub. No. US 2015/0376609, which is hereby incorporated by reference in its entirety.

FIG. 12 shows an example of a microfluidic channel structure 1400 for delivering barcode carrying beads to droplets. The channel structure 1400 can include channel segments 1401, 1402, 1404, 1406 and 1408 communicating at a channel junction 1410. In operation, the channel segment 1401 may transport an aqueous fluid 1412 that includes a plurality of beads 1414 (e.g., with nucleic acid molecules, e.g., nucleic acid barcode molecules or barcoded oligonucleotides, molecular tags) along the channel segment 1401 into junction 1410. The plurality of beads 1414 may be sourced from a suspension of beads. For example, the channel segment 1401 may be connected to a reservoir comprising an aqueous suspension of beads 1414. The channel segment 1402 may transport the aqueous fluid 1412 that includes a plurality of biological particles 1416 along the channel segment 1402 into junction 1410. The plurality of biological particles 1416 may be sourced from a suspension of biological particles. For example, the channel segment 1402 may be connected to a reservoir comprising an aqueous suspension of biological particles 1416. In some instances, the aqueous fluid 1412 in either the first channel segment 1401 or the second channel segment 1402, or in both segments, can include one or more reagents, as further described below. A second fluid 1418 that is immiscible with the aqueous fluid 1412 (e.g., oil) can be delivered to the junction 1410 from each of channel segments 1404 and 1406. Upon meeting of the aqueous fluid 1412 from each of channel segments 1401 and 1402 and the second fluid 1418 from each of channel segments 1404 and 1406 at the channel junction 1410, the aqueous fluid 1412 can be partitioned as discrete droplets 1420 in the second fluid 1418 and flow away from the junction 1410 along channel segment 1408. The channel segment 1408 may deliver the discrete droplets to an outlet reservoir fluidly coupled to the channel segment 1408, where they may be harvested. As an alternative, the channel segments 1401 and 1402 may meet at another junction upstream of the junction 1410. At such junction, beads and biological particles may form a mixture that is directed along another channel to the junction 1410 to yield droplets 1420. The mixture may provide the beads and biological particles in an alternating fashion, such that, for example, a droplet comprises a single bead and a single biological particle.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

As described above, FIG. 13 shows an example of a microfluidic channel structure for the controlled partitioning of beads into discrete droplets. A channel structure 1400 can include a channel segment 1402 communicating at a channel junction 1406 (or intersection) with a reservoir 1404. The reservoir 1404 can be a chamber. Any reference to “reservoir,” as used herein, can also refer to a “chamber.” In operation, an aqueous fluid 1408 that includes suspended beads 1412 may be transported along the channel segment 1402 into the junction 1406 to meet a second fluid 1410 that is immiscible with the aqueous fluid 1408 in the reservoir 1404 to create droplets 1416, 1418 of the aqueous fluid 1408 flowing into the reservoir 1404. At the junction 1406 where the aqueous fluid 1408 and the second fluid 1410 meet, droplets can form based on factors such as the hydrodynamic forces at the junction 1406, flow rates of the two fluids 1408, 1410, fluid properties, and certain geometric parameters (e.g., w, h₀, α, etc.) of the channel structure 1400. A plurality of droplets can be collected in the reservoir 1404 by continuously injecting the aqueous fluid 1408 from the channel segment 1402 through the junction 1406.

Referring to FIG. 13, in some instances, the aqueous fluid 208 can have a substantially uniform concentration or frequency of beads 212. The beads 212 can be introduced into the channel segment 202 from a separate channel (not shown in FIG. 13). The frequency of beads 212 in the channel segment 202 may be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

Referring to FIG. 13, in some instances, the aqueous fluid 1408 in the channel segment 1402 can comprise biological particles. In some instances, the aqueous fluid 1408 can have a substantially uniform concentration or frequency of biological particles. As with the beads, the biological particles can be introduced into the channel segment 1402 from a separate channel. The frequency or concentration of the biological particles in the aqueous fluid 1408 in the channel segment 1402 may be controlled by controlling the frequency in which the biological particles are introduced into the channel segment 1402 and/or the relative flow rates of the fluids in the channel segment 1402 and the separate channel. In some instances, the biological particles can be introduced into the channel segment 1402 from a plurality of different channels, and the frequency controlled accordingly. In some instances, a first separate channel can introduce beads and a second separate channel can introduce biological particles into the channel segment 1402. The first separate channel introducing the beads may be upstream or downstream of the second separate channel introducing the biological particles.

Referring to FIG. 13, the second fluid 1410 can comprise an oil, such as a fluorinated oil, that includes a fluorosurfactant for stabilizing the resulting droplets, for example, inhibiting subsequent coalescence of the resulting droplets.

Referring to FIG. 13, in some instances, the second fluid 1410 may not be subjected to and/or directed to any flow in or out of the reservoir 1404. For example, the second fluid 1410 may be substantially stationary in the reservoir 1404. In some instances, the second fluid 1410 may be subjected to flow within the reservoir 1404, but not in or out of the reservoir 1404, such as via application of pressure to the reservoir 1404 and/or as affected by the incoming flow of the aqueous fluid 1408 at the junction 1406. Alternatively, the second fluid 1410 may be subjected and/or directed to flow in or out of the reservoir 1404. For example, the reservoir 1404 can be a channel directing the second fluid 1410 from upstream to downstream, transporting the generated droplets.

Systems and methods for controlled partitioning are described further in PCT/US2018/047551, which is hereby incorporated by reference in its entirety.

A cell bead can contain a biological particle (e.g., a cell) or macromolecular constituents (e.g., RNA, DNA, proteins, etc.) of a biological particle. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Cell beads, and exemplary methods for creating cell beads, are described in U.S. Patent Application Pub. No. US 2015/0376609 and PCT/US2018/016019, which are hereby incorporated by reference in their entirety.

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

The solid support may be a bead. A solid support, e.g., a bead, may be porous, non-porous, hollow (e.g., a microcapsule), solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Nucleic acid barcode molecules can be delivered to a partition via a microcapsule. A microcapsule, in some instances, can comprise a bead. Beads are described in further detail below.

In some cases, nucleic acid barcode molecules can be initially associated with the microcapsule and then released from the microcapsule. Release of the nucleic acid barcode molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the nucleic acid barcode molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

Methods and systems for partitioning barcode carrying beads into droplets are provided herein, and in in US. Patent Publication Nos. 2019/0367997 and 2019/0064173, and International Application No. PCT/US20/17785, each of which is herein entirely incorporated by reference for all purposes.

A bead may be porous, non-porous, solid, semi-solid, semi-fluidic, fluidic, and/or a combination thereof. In some instances, a bead may be dissolvable, disruptable, and/or degradable. Degradable beads, as well as methods for degrading beads, are described in PCT/US2014/044398, which is hereby incorporated by reference in its entirety. In some cases, any combination of stimuli, e.g., stimuli described in PCT/US2014/044398 and US Patent Application Pub. No. 2015/0376609, hereby incorporated by reference in its entirety, may trigger degradation of a bead. For example, a change in pH may enable a chemical agent (e.g., DTT) to become an effective reducing agent.

In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. See, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

In some cases, a plurality of nucleic acid barcode molecules may be attached to a bead. The nucleic acid barcode molecules may be attached directly or indirectly to the bead. In some cases, the nucleic acid barcode molecules may be covalently linked to the bead. In some cases, the nucleic acid barcode molecules may be covalently linked to the bead via a linker. In some cases, the linker may be a degradable linker. In some cases, the linker may comprise a labile bond configured to release one or more nucleic acid barcode molecules of said plurality of nucleic acid barcode molecules. In some cases, the labile bond may comprise a disulfide linkage.

Activation of disulfide linkages within a bead can be controlled such that only a small number of disulfide linkages are activated. Methods of controlling activation of disulfide linkages within a bead are described in PCT/US2014/044398, which is hereby incorporated by reference in its entirety.

In some cases, a bead may comprise an acrydite moiety, which in certain aspects may be used to attach one or more nucleic acid molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, which is hereby incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

Other non-limiting examples of labile bonds that may be coupled to a precursor or bead are described in PCT/US2014/044398, which is hereby incorporated by reference in its entirety. A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. See, e.g., PCT/US2014/044398, which is hereby incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively or in addition, species may be partitioned in a partition (e.g., droplet) during or subsequent to partition formation. Such species may include, without limitation, the abovementioned species that may also be encapsulated in a bead.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In accordance with certain aspects, biological particles and/or biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning biological particles with reagents. Such systems and methods are described in U.S. Patent Publication No. US/20190367997, which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned microcapsule. For example, in some cases, a chemical stimulus may be co-partitioned along with an encapsulated biological particle to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative examples, this may be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particle, such as endonucleases to fragment an biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some cases, template switching can be used to increase the length of a cDNA. In some cases, template switching can be used to append a predefined nucleic acid sequence to the cDNA. Template switching is further described in PCT/US2017/068320, which is hereby incorporated by reference in its entirety. Template switching oligonucleotides may comprise a hybridization region and a template region. Template switching oligonucleotides are further described in PCT/US2017/068320, which is hereby incorporated by reference in its entirety.

Any of the reagents described in this disclosure may be encapsulated in, or otherwise coupled to, a microcapsule, droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles. In some aspects, this is performed by co-partitioning the individual biological particle or groups of biological particles with the unique identifiers.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

A nucleic acid barcode molecule may contain one or more barcode sequences. A plurality of nucleic acid barcode molecules may be coupled to a bead. The one or more barcode sequences may include sequences that are the same for all nucleic acid molecules coupled to a given bead and/or sequences that are different across all nucleic acid molecules coupled to the given bead. The nucleic acid molecule may be incorporated into the bead.

Nucleic acid barcode molecules can comprise one or more functional sequences for coupling to an analyte or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

In some cases, the nucleic acid barcode molecule can further comprise a unique molecular identifier (UMI). In some cases, the nucleic acid barcode molecule can comprise one or more functional sequences, for example, for attachment to a sequencing flow cell, such as, for example, a P5 sequence (or a portion thereof) for Illumina® or next-generational sequencing. In some cases, the nucleic acid barcode molecule or derivative thereof (e.g., oligonucleotide or polynucleotide generated from the nucleic acid molecule) can comprise another functional sequence, such as, for example, a P7 sequence (or a portion thereof) for attachment to a sequencing flow cell for Illumina® or next-generational sequencing. In some cases, the nucleic acid barcode molecule can comprise an R1 primer sequence for Illumina® or next generational sequencing. In some cases, the nucleic acid barcode molecule can comprise an R2 primer sequence for Illumina® or next generational sequencing.

In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid barcode molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

FIG. 14 illustrates an example of a barcode-molecule-carrying bead 1500. A nucleic acid barcode molecule 1502 can be coupled to a bead 1504 by a releasable linkage 1506, such as, for example, a disulfide linker. The same bead 1504 may be coupled (e.g., via releasable linkage) to one or more other nucleic acid barcode molecules 1518, 1520. The nucleic acid barcode molecule 1502 may be, or may comprise a barcode. As noted elsewhere herein, the structure of the barcode may comprise a number of sequence elements. The nucleic acid barcode molecule 1502 may comprise a functional sequence 1508 that may be used in subsequent processing. For example, the functional sequence 1508 may include one or more of a sequencer specific flow cell attachment sequence (e.g., a P5 sequence for ILLUMINA sequencing systems) and a sequencing primer sequence (e.g., a R1 primer for ILLUMINA sequencing systems). The nucleic acid barcode molecule 1502 may comprise a barcode sequence 1510 for use in barcoding the sample (e.g., DNA, RNA, protein, antibody, etc.).

In some cases, the barcode sequence 1510 can be bead-specific such that the barcode sequence is common to all nucleic acid barcode molecules (e.g., including nucleic acid barcode molecule 1502) coupled to the same bead 1504. Alternatively, or in addition, the barcode sequence 1510 can be partition-specific such that the barcode sequence is common to all nucleic acid barcode molecules coupled to one or more beads that are partitioned into the same partition.

The nucleic acid barcode molecule 1502 may comprise a specific priming sequence 1512, such as an mRNA specific priming sequence (e.g., poly-T sequence), a targeted priming sequence, and/or a random priming sequence. The nucleic acid barcode molecule 1502 may comprise an anchoring sequence 1514 to ensure that the specific priming sequence 1512 hybridizes at the sequence end (e.g., of the mRNA). For example, the anchoring sequence 1514 can include a random short sequence of nucleotides, such as a 1-mer, 2-mer, 3-mer or longer sequence, which can ensure that a poly-T segment is more likely to hybridize at the sequence end of the poly-A tail of the mRNA.

Referring to FIG. 14, the nucleic acid barcode molecule 1502 may comprise a unique molecular identifying sequence 1516 (e.g., unique molecular identifier (UMI)).

In some cases, the unique molecular identifying sequence 1516 may comprise from about 5 to about 8 nucleotides.

In some embodiments, the unique molecular identifying sequence 1516 may be of any length.

A UMI sequence may comprise two or more, or three or more, or four or more, or five or more, or six or more, or seven or more, or eight or more, or nine or more, or ten or more nucleotides.

A UMI sequence may comprise from 2 to 6, or 2 to 8, or 2 to 10, or 2 to 12, or 2 to 20, or 2 to 30, or 2 to 50 nucleotides.

A UMI sequence may comprise 2, or 3, or 4, or 5, or 6, or 7, or 8, or 9, or 10, or 11, or 12, or 13, or 14, or 15 nucleotides.

The unique molecular identifying sequence 1516 may be the same or different for each nucleic acid barcode molecule (e.g., 1502, 1518, 1520, etc.) coupled to a single bead (e.g., bead 1504).

In some cases, the unique molecular identifying sequence 1516 may be a random sequence (e.g., such as a random N-mer sequence).

In some embodiments, a UMI may provide a unique identifier of an mRNA molecule that was captured, in order to allow quantitation of the number of such mRNA molecules originally expressed in a cell, sample, culture, measurement, or system.

As will be appreciated, although FIG. 14 shows three nucleic acid barcode molecules 1502, 1518, 1520 coupled to the surface of the bead 1504, an individual bead may be coupled to essentially any number of individual nucleic acid barcode molecules.

For example, a bead or support may be coupled to from one to ten, or from 1 to 100, or from one to one thousand, or from one to ten thousand, or from one to 100,000, or from one to one million, or from one to one billion, or from one to many billions or more of nucleic acid barcode molecules.

The barcode sequences for the individual nucleic acid barcode molecules attached to a single bead or support can comprise common sequence segments, or relatively common sequence segments (e.g., 1508, 1510, 1512, etc.) and variable or unique sequence segments (e.g., 1516) between different individual nucleic acid barcode molecules.

Referring to FIG. 14, a biological particle (e.g., cell, DNA, RNA, etc.) can be co-partitioned along with a barcode bearing bead 1504. The nucleic acid barcode molecules 1502, 1518, 1520 can be released from the bead 1504 in the partition. By way of example, in the context of analyzing sample RNA, the poly-T segment (e.g., 1512) of one of the released nucleic acid barcode molecules (e.g., 1502) can hybridize to the poly-A tail of a mRNA molecule. Reverse transcription may result in a cDNA transcript of the mRNA, but which transcript includes each of the sequence segments 1508, 1510, 1516 of the nucleic acid barcode molecule 1502. Because the nucleic acid barcode molecule 1502 comprises an anchoring sequence 1514, it will more likely hybridize to and prime reverse transcription at the sequence end of the poly-A tail of the mRNA. cDNA transcripts of the individual mRNA molecules from any given partition may include a common barcode sequence segment 1510. For example, within any given partition, all of the cDNA transcripts of the individual mRNA molecules may include a common barcode sequence segment 1510.

Referring to FIG. 14, the transcripts made from the different mRNA molecules within a given partition may vary at the unique molecular identifying sequence 1512 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions may be collected, pooled, and subjected to further processing (e.g., reverse transcription, adapter attachment, amplification, clean up, sequencing). In other instances, one or more of the processing methods, e.g., reverse transcription, may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise 2, or 3, or 4, or 5, or 6, or 7, or 8, or 9, or 10 or more different capture sequences or binding sequences configured to bind to different respective capture sequences or binding sequences, respectively.

In some embodiments, a bead may comprise less than 18, or 16, or 14, or 12, or 10, or 9, or 8, or 7, or 6, or 5, or 4, or 3, or 2 different capture sequences or binding sequences configured to bind to different respective capture sequences or binding sequences.

In certain embodiments, a bead may comprise 2, or 3, or 4, or 5, or 6, or 7, or 8, or 9, or 10 different capture sequences or binding sequences configured to bind to different respective capture sequences or binding sequences.

In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, a biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

FIG. 14 illustrates an example of a barcode carrying bead 1600. A nucleic acid barcode molecule 1605, such as an oligonucleotide, can be coupled to a bead 1604 by a releasable linkage 1606, such as, for example, a disulfide linker. The nucleic acid barcode molecule 1605 may comprise a first capture sequence 1660. The same bead 1604 may be coupled (e.g., via releasable linkage) to one or more other nucleic acid molecules 1603, 1607 comprising other capture sequences. The nucleic acid barcode molecule 1605 may be, or may contain a barcode. As noted elsewhere herein, the structure of the barcode may comprise a number of sequence elements, such as a functional sequence 1608 (e.g., flow cell attachment sequence, sequencing primer sequence, etc.), a barcode sequence 1610 (e.g., bead-specific sequence common to bead, partition-specific sequence common to partition, etc.), and a unique molecular identifier 1612 (e.g., unique sequence within different molecules attached to the bead), or partial sequences thereof. The capture sequence 1660 may be configured to attach to a corresponding capture sequence 1665. In some instances, the corresponding capture sequence 1665 may be coupled to another molecule that may be an analyte or an intermediary carrier.

For example, as illustrated in FIG. 14, the corresponding capture sequence 1665 is coupled to a guide RNA molecule 1662 comprising a target sequence 1664, wherein the target sequence 1664 is configured to attach to the analyte. Another oligonucleotide molecule 1607 attached to the bead 1604 comprises a second capture sequence 1680 which is configured to attach to a second corresponding capture sequence 1685.

As illustrated in FIG. 14, the second corresponding capture sequence 1685 is coupled to an antibody 1682. In some cases, the antibody 1682 may have binding specificity to an analyte (e.g., surface protein). Alternatively, the antibody 1682 may not have binding specificity.

Another oligonucleotide molecule 1603 attached to the bead 1604 comprises a third capture sequence 1670 which is configured to attach to a second corresponding capture sequence 1675. As illustrated in FIG. 14, the third corresponding capture sequence 1675 is coupled to a molecule 1672. The molecule 1672 may or may not be configured to target an analyte. The other oligonucleotide molecules 1603, 1607 may comprise the other sequences (e.g., functional sequence, barcode sequence, UMI, etc.) described with respect to oligonucleotide molecule 1605. While a single oligonucleotide molecule comprising each capture sequence is illustrated in FIG. 14, it will be appreciated that, for each capture sequence, the bead may comprise a set of one or more oligonucleotide molecules each comprising the capture sequence.

For example, the bead may comprise any number of sets of one or more different capture sequences. Alternatively, or in addition, the bead 404 may comprise other capture sequences. Alternatively, or in addition, the bead 404 may comprise fewer types of capture sequences (e.g., two capture sequences). Alternatively or in addition, the bead 1604 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

Referring to FIG. 14, the moieties 1672, 1662, and 1682 can be considered labeling agents. Any of the labeling agents described herein may be attached (either directly, e.g., covalently attached, or indirectly) to a reporter oligonucleotide. The moieties 1603, 1605, and 1607 can also be considered reporter oligonucleotides. Exemplary reporter oligonucleotides are described herein.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein.

Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein.

In another example, entrained species may be released from beads through osmotic pressure differences due to, for example, changing chemical environments. See, e.g., PCT/US2014/044398, which is hereby incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. See, e.g., PCT/US2014/044398, which is hereby incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

A nucleic acid barcode sequence of this disclosure may comprise any length.

In some embodiments, nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads are provided that each include large numbers of the above described nucleic acid barcode molecules releasably attached to the beads, where all of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, each bead can be provided with large numbers of nucleic acid (e.g., oligonucleotide) barcode molecules attached. In particular, the number of attached nucleic acid barcode molecules on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more.

In certain embodiments, the number of attached nucleic acid barcode molecules on an individual bead can be from 10³ to 10¹², or from 10⁶ to 10¹², or from 10⁹ to 10¹².

Nucleic acid molecules of a given bead can include identical (i.e. common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set. In some embodiments, such different barcode sequences can be associated with a given bead.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules or more.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a microcapsule, droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, nucleic acid barcode molecules, enzymes, adapters, beads, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a microcapsule or bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the microcapsule or bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

Reagents may be loaded into a well either sequentially or concurrently. In some cases, reagents are introduced to the device either before or after a particular operation. In some cases, reagents (which may be provided, in certain instances, in microcapsules, droplets, or beads) are introduced sequentially such that different reactions or operations occur at different steps. The reagents (or microcapsules, droplets, or beads) may also be loaded at operations interspersed with a reaction or operation step. For example, microcapsules (or droplets or beads) comprising reagents for fragmenting polynucleotides (e.g., restriction enzymes) and/or other enzymes (e.g., transposases, ligases, polymerases, etc.) may be loaded into the well or plurality of wells, followed by loading of microcapsules, droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson.

A droplet or bead may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecules may be attached to a droplet or bead that has been partitioned into the well. The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. In some instances, the nucleic acid barcode molecules may be releasable from the bead or droplet. For instance, the nucleic acid barcode molecules may comprise a chemical cross-linker which may be cleaved upon application of a stimulus (e.g., photo-, magnetic, chemical, biological, stimulus). The nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In some instances nucleic acid barcode molecules attached to a bead or droplet in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to: dynamic live-cell tracking, cell-cell interactions (when two or more cells are co-partitioned), cell proliferation, etc. Alternatively or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

FIG. 15 schematically illustrates an example of a microwell array. The array can be contained within a substrate 1700. The substrate 1700 comprises a plurality of wells 1702. The wells 1702 may be of any size or shape, and the spacing between the wells, the number of wells per substrate, as well as the density of the wells on the substrate 1700 can be modified, depending on the particular application. In one such example application, a sample molecule 1706, which may comprise a cell or cellular components (e.g., nucleic acid molecules) is co-partitioned with a bead 1704, which may comprise a nucleic acid barcode molecule coupled thereto. The wells 1702 may be loaded using gravity or other loading technique (e.g., centrifugation, liquid handler, acoustic loading, optoelectronic, etc.). In some instances, at least one of the wells 1702 contains a single sample molecule 1706 (e.g., cell) and a single bead 1704.

FIG. 16 schematically shows an example workflow for processing nucleic acid molecules within a sample. A substrate 1800 comprising a plurality of microwells 1802 may be provided. A sample 1806 which may comprise a cell, cell bead, cellular components or analytes (e.g., proteins and/or nucleic acid molecules) can be co-partitioned, in a plurality of microwells 1802, with a plurality of beads 1804 comprising nucleic acid barcode molecules. During process 1810, the sample 1806 may be processed within the partition. For instance, in the case of live cells, the cell may be subjected to conditions sufficient to lyse the cells and release the analytes contained therein. In process 1820, the bead 1804 may be further processed. By way of example, processes 1821 and 1822 schematically illustrate different workflows, depending on the properties of the bead 1804.

Referring to FIG. 16, in 1821, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 1830, the beads 1804 from multiple wells 1802 may be collected and pooled. Further processing may be performed in process 1840. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1850, further characterization, such as sequencing may be performed to generate sequencing reads. The sequencing reads may yield information on individual cells or populations of cells, which may be represented visually or graphically, e.g., in a plot 1855.

Referring to FIG. 16, in 1822, the bead comprises nucleic acid barcode molecules that are releasably attached thereto, as described below. The bead may degrade or otherwise release the nucleic acid barcode molecules into the well 1802; the nucleic acid barcode molecules may then be used to barcode nucleic acid molecules within the well 1802. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 1850, further characterization, such as sequencing may be performed to generate sequencing reads. The sequencing reads may yield information on individual cells or populations of cells, which may be represented visually or graphically, e.g., in a plot 1855.

The present disclosures provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labeling agents capable of binding to or otherwise coupling to one or more cell features may be used to characterize biological particles and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have a first reporter oligonucleotide coupled thereto, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell feature labeling agents may be provided, where the respective cell feature labeling agents are associated with nucleic acid reporter molecules, such that a different reporter oligonucleotide sequence is associated with each labeling agent capable of binding to a specific cell feature. In some aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labeling agents capable of binding to or otherwise coupling to one or more biological particles may be used to characterize an biological particle as belonging to a particular set of biological particles. For example, labeling agents may be used to label a sample of cells or a group of cells. In this way, a group of cells may be labeled as different from another group of cells. In an example, a first group of cells may originate from a first sample and a second group of cells may originate from a second sample. Labeling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling an biological particle may comprise subjecting the antibody-linked barcode molecule or the epitope binding fragment-linked barcode molecule to conditions suitable for binding the antibody to a molecule present on a surface of the biological particle. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into an biological particle. Labeling biological particles may comprise delivering the CPP conjugated oligonucleotide into a cell and/or cell bead by the cell-penetrating peptide. A cell-penetrating peptide that can be used in the methods provided herein can comprise at least one non-functional cysteine residue, which may be either free or derivatized to form a disulfide link with an oligonucleotide that has been modified for such linkage. Non-limiting examples of cell-penetrating peptides that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The cell-penetrating peptide may be an arginine-rich peptide transporter. The cell-penetrating peptide may be Penetratin or the Tat peptide.

In another example, a reporter oligonucleotide may be coupled to a fluorophore or dye, and labeling cells may comprise subjecting the fluorophore-linked barcode molecule to conditions suitable for binding the fluorophore to the surface of the biological particle. In some instances, fluorophores can interact strongly with lipid bilayers and labeling biological particles may comprise subjecting the fluorophore-linked barcode molecule to conditions such that the fluorophore binds to or is inserted into a membrane of the biological particle. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide may be coupled to a lipophilic molecule, and labeling biological particles may comprise delivering the nucleic acid barcode molecule to a membrane of the biological particle or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and biological particle may be such that the biological particle retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labeling agents, that may be labeling agents to a broad panel of different cell features, e.g., receptors, proteins, etc., and which include their associated reporter oligonucleotides. Unbound labeling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labeling agents and their known, associated reporter oligonucleotides.

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labeling agents may be associated with a particular cell feature as well as be used to identify analytes as originating from a particular biological particle, population, or sample. The biological particles may be incubated with a plurality of libraries and a given biological particle may comprise multiple labeling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labeling agents may allow multi-analyte, multiplexed analyses to be performed.

In some instances, these reporter oligonucleotides may comprise nucleic acid barcode sequences that permit identification of the labeling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labeling agents. Commercially available kits, such as those from THUNDERLINK and ABCAM, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

FIG. 17 describes exemplary labeling agents (1110, 1120, 1130) comprising reporter oligonucleotides (1140) attached thereto. Labeling agent 1110 (e.g., any of the labeling agents described herein) is attached (either directly, e.g., covalently attached, or indirectly) to reporter oligonucleotide 1140. Reporter oligonucleotide 1140 may comprise barcode sequence 1142 that identifies labeling agent 1110. Reporter oligonucleotide 1140 may also comprise one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 17, in some instances, reporter oligonucleotide 1140 conjugated to a labeling agent (e.g., 1110, 1120, 1130) comprises a functional sequence 1141 (e.g., a primer sequence), a barcode sequence that identifies the labeling agent (e.g., 1110, 1120, 1130), and functional sequence 1143. Functional sequence 1143 can be a reporter capture handle sequence configured to hybridize to a complementary sequence, such as a complementary sequence present on a nucleic acid barcode molecule 1190 (not shown), such as those described elsewhere herein. In some instances, nucleic acid barcode molecule 1190 is attached to a support (e.g., a bead, such as a gel bead), such as those described elsewhere herein. For example, nucleic acid barcode molecule 1190 may be attached to the support via a releasable linkage (e.g., comprising a labile bond), such as those described elsewhere herein. In some instances, reporter oligonucleotide 1140 comprises one or more additional functional sequences, such as those described above.

In some instances, the labeling agent 1110 is a protein or polypeptide (e.g., an antigen or prospective antigen) comprising reporter oligonucleotide 1140. Reporter oligonucleotide 1140 comprises barcode sequence 1142 that identifies polypeptide 1110 and can be used to infer the presence of an analyte, e.g., a binding partner of polypeptide 1110 (i.e., a molecule or compound to which polypeptide 1110 can bind). In some instances, the labeling agent 1110 is a lipophilic moiety (e.g., cholesterol) comprising reporter oligonucleotide 1140, where the lipophilic moiety is selected such that labeling agent 1110 integrates into a membrane of a cell or nucleus. Reporter oligonucleotide 1140 comprises barcode sequence 1142 that identifies lipophilic moiety 1110 which in some instances is used to tag cells (e.g., groups of cells, cell samples, etc.) and may be used for multiplex analyses as described elsewhere herein. In some instances, the labeling agent is an antibody 1120 (or an epitope binding fragment thereof) comprising reporter oligonucleotide 1140. Reporter oligonucleotide 1140 comprises barcode sequence 1142 that identifies antibody 1120 and can be used to infer the presence of, e.g., a target of antibody 1120 (i.e., a molecule or compound to which antibody 1120 binds). In other embodiments, labeling agent 1130 comprises an MHC molecule 1131 comprising peptide 1132 and reporter oligonucleotide 1140 that identifies peptide 1132. In some instances, the MHC molecule is coupled to a support 1133. In some instances, support 1133 may be a polypeptide, such as streptavidin, or a polysaccharide, such as dextran. In some instances, reporter oligonucleotide 1140 may be directly or indirectly coupled to MHC labeling agent 1130 in any suitable manner. For example, reporter oligonucleotide 1140 may be coupled to MHC molecule 1131, support 1133, or peptide 1132. In some embodiments, labeling agent 1130 comprises a plurality of MHC molecules, (e.g. is an MHC multimer, which may be coupled to a support (e.g., 1133)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labeling agents, including antibody and MHC-based labeling agents, reporter oligonucleotides, and methods of use, see, e.g., U.S. Pat. No. 10,550,429 and U.S. Pat. Pub. 20190367969, each of which is herein entirely incorporated by reference for all purposes.

Referring to FIG. 18A, in an instance where cells are labeled with labeling agents, capture sequence 1223 may be complementary to an adapter sequence of a reporter oligonucleotide. Cells may be contacted with one or more reporter oligonucleotide 1220 conjugated labeling agents 1210 (e.g., polypeptide, antibody, or others described elsewhere herein). In some cases, the cells may be further processed prior to barcoding. For example, such processing steps may include one or more washing and/or cell sorting steps. In some instances, a cell that is bound to labeling agent 1210 which is conjugated to oligonucleotide 1220 and support 1230 (e.g., a bead, such as a gel bead) comprising nucleic acid barcode molecule 1290 is partitioned into a partition amongst a plurality of partitions (e.g., a droplet of a droplet emulsion or a well of a microwell array). In some instances, the partition comprises at most a single cell bound to labeling agent 1210. In some instances, reporter oligonucleotide 1220 conjugated to labeling agent 1210 (e.g., polypeptide, an antibody, pMHC molecule such as an MHC multimer, etc.) comprises a first adapter sequence 1211 (e.g., a primer sequence), a barcode sequence 1212 that identifies the labeling agent 1210 (e.g., the polypeptide, antibody, or peptide of a pMHC molecule or complex), and an capture handle sequence 1213. Capture handle sequence 1213 may be configured to hybridize to a complementary sequence, such as a capture sequence 1223 present on a nucleic acid barcode molecule 1290. In some instances, oligonucleotide 1220 comprises one or more additional functional sequences, such as those described elsewhere herein.

Barcoded nucleic may be generated (e.g., via a nucleic acid reaction, such as nucleic acid extension or ligation) from the constructs described in FIGS. 18A-C. For example, capture handle sequence 1213 may then be hybridized to complementary sequence, such as capture sequence 1223 to generate (e.g., via a nucleic acid reaction, such as nucleic acid extension or ligation) a barcoded nucleic acid molecule comprising cell (e.g., partition specific) barcode sequence 1222 (or a reverse complement thereof) and reporter barcode sequence 1212 (or a reverse complement thereof). In some embodiments, the nucleic acid barcode molecule 1290 (e.g., partition-specific barcode molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labeling agents described herein) may be performed. For example, the workflow may comprise a workflow as generally depicted in any of FIGS. 18A-C, or a combination of workflows for an individual analyte, as described elsewhere herein. For example, by using a combination of the workflows as generally depicted in FIGS. 18A-C, multiple analytes can be analyzed.

In some instances, analysis of an analyte (e.g. a nucleic acid, a polypeptide, a carbohydrate, a lipid, etc.) comprises a workflow using moieties generally depicted in FIG. 18A. A nucleic acid barcode molecule 1290 may be co-partitioned with the one or more analytes. In some instances, nucleic acid barcode molecule 1290 is attached to a support 1230 (e.g., a bead, such as a gel bead), such as those described elsewhere herein. For example, nucleic acid barcode molecule 1290 may be attached to support 1230 via a releasable linkage 1240 (e.g., comprising a labile bond), such as those described elsewhere herein. Nucleic acid barcode molecule 1290 may comprise a functional sequence 1221 and optionally comprise other additional sequences, for example, a barcode sequence 1222 (e.g., common barcode, partition-specific barcode, or other functional sequences described elsewhere herein), and/or a UMI sequence (not shown). The nucleic acid barcode molecule 1290 may comprise a capture sequence 1223 that may be complementary to another nucleic acid sequence, such that it may hybridize to a particular sequence, e.g., capture handle sequence 1213.

For example, capture sequence 1223 may comprise a poly-T sequence and may be used to hybridize to mRNA. Referring to FIG. 18C, in some embodiments, nucleic acid barcode molecule 1290 comprises capture sequence 1223 complementary to a sequence of RNA molecule 1260 from a cell. In some instances, capture sequence 1223 comprises a sequence specific for an RNA molecule. Capture sequence 1223 may comprise a known or targeted sequence or a random sequence. In some instances, a nucleic acid extension reaction may be performed, thereby generating a barcoded nucleic acid product comprising capture sequence 1223, the functional sequence 1221, barcode sequence 1222, any other functional sequence, and a sequence corresponding to the RNA molecule 1260.

In another example, capture sequence 1223 may be complementary to an overhang sequence or an adapter sequence that has been appended to an analyte. For example, referring to FIG. 18B, panel 1201, in some embodiments, primer 1250 comprises a sequence complementary to a sequence of nucleic acid molecule 1260 (such as an RNA encoding for a BCR sequence) from an biological particle. In some instances, primer 1250 comprises one or more sequences 1251 that are not complementary to RNA molecule 1260. Sequence 1251 may be a functional sequence as described elsewhere herein, for example, an adapter sequence, a sequencing primer sequence, or a sequence the facilitates coupling to a flow cell of a sequencer. In some instances, primer 1250 comprises a poly-T sequence. In some instances, primer 1250 comprises a sequence complementary to a target sequence in an RNA molecule. In some instances, primer 1250 comprises a sequence complementary to a region of an immune molecule, such as the constant region of a TCR or BCR sequence. Primer 1250 is hybridized to nucleic acid molecule 1260 and complementary molecule 1270 is generated (see Panel 1202). For example, complementary molecule 1270 may be cDNA generated in a reverse transcription reaction. In some instances, an additional sequence may be appended to complementary molecule 1270. For example, the reverse transcriptase enzyme may be selected such that several non-templated bases 1280 (e.g., a poly-C sequence) are appended to the cDNA. In another example, a terminal transferase may also be used to append the additional sequence. Nucleic acid barcode molecule 1290 comprises a sequence 1224 complementary to the non-templated bases, and the reverse transcriptase performs a template switching reaction onto nucleic acid barcode molecule 1290 to generate a barcoded nucleic acid molecule comprising cell (e.g., partition specific) barcode sequence 1222 (or a reverse complement thereof) and a sequence of complementary molecule 1270 (or a portion thereof). In some instances, sequence 1223 comprises a sequence complementary to a region of an immune molecule, such as the constant region of a TCR or BCR sequence. Sequence 1223 is hybridized to nucleic acid molecule 1260 and a complementary molecule 1270 is generated. For example complementary molecule 1270 may be generated in a reverse transcription reaction generating a barcoded nucleic acid molecule comprising cell (e.g., partition specific) barcode sequence 1222 (or a reverse complement thereof) and a sequence of complementary molecule 1270 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, each of which applications is herein entirely incorporated by reference for all purposes.

The methods described herein may comprise templated ligation. A templated ligation process may comprise contacting a nucleic acid molecule (e.g., an RNA molecule) with a probe molecule. The probe molecule may interact with one or more other probe molecules, for example, comprising a barcode sequence, to generate a probe-barcode complex. An extension reaction may be performed on at least a portion of the probe-barcode complex to generate a nucleic acid product that comprises the barcode sequence and is associated with a sequence of the nucleic acid molecule. Beneficially, the methods described herein may allow barcoding of the nucleic acid molecule without performing reverse transcription on the nucleic acid molecule. The methods herein may comprise ligation-mediated reactions.

A method may comprise contacting a nucleic acid molecule (e.g., an RNA molecule) with a first probe molecule, comprising a first sequence and a second sequence, under conditions sufficient for the first sequence to hybridize to a sequence of the nucleic acid molecule. A second probe molecule comprising a third sequence may hybridize to the second sequence of the first probe molecule. The first probe or the second probe molecule may comprise a barcode sequence (e.g., as described herein). For example, the second probe molecule may be a nucleic acid molecule (e.g., as described herein). In some cases, a splint molecule may be used to link the first and second probe molecules. For example, a fourth sequence of the splint molecule may hybridize to the second sequence of the first probe molecule and a fifth sequence of the splint molecule may hybridize to the third sequence of the second probe molecule.

In another example, a first probe molecule with a first reactive moiety and a second probe molecule with a second reactive moiety may be used. A first sequence of the first probe molecule may hybridize to a first sequence of a nucleic acid molecule and a second sequence of the second probe molecule may hybridize to a second sequence of the nucleic acid molecule. The first and second sequences of the nucleic acid molecule may be adjacent or may be separated by a gap of one or more nucleotides, which gap may optionally be filled (e.g., using a polymerase). The first reactive moiety of the first probe molecule and the second reactive moiety of the second probe molecule may be subjected to conditions sufficient for the first and second reactive moieties to react to provide a linking moiety. For example, a click chemistry reaction involving an alkyne moiety and an azide moiety may be used to provide a triazole linking moiety. In other examples, an iodide moiety may be chemically ligated to a phosphorothioate moiety to form a phosphorothioate bond, an acid may be ligated to an amine to form an amide bond, or a phosphate may be ligated to an amine to form a phosphoramidate bond. In some cases, the probes may be subjected to an enzymatic ligation reaction, using a ligase, e.g., SplintR ligases, T4 ligases, KOD ligases, PBCV1 enzymes, etc. to form a probe-linked nucleic acid molecule. Where the two probes are non-adjacent, gap regions between the probes may be filled prior to ligation. In some instances, ribonucleotides or deoxyribonucleotides are ligated between the first and second probes.

Prior to, in parallel, or subsequent to linking of the first and second probe molecules (e.g., via reaction between their respective reactive moieties), a third probe molecule (e.g., a nucleic acid barcode molecule) may be subjected to conditions sufficient to hybridize to a third sequence of the first probe molecule. The third probe molecule may comprise a barcode sequence. In some cases, a splint molecule may be used to link the first and third probe molecules. In some cases, the first and second probe molecules may be linked to one another such that a loop or “padlock” is formed after hybridization of the first sequence of the first probe molecule to the first sequence of the nucleic acid molecule and the second sequence of the second probe molecule to the second sequence of the nucleic acid molecule. A linkage between the first and second probe molecules may be generated after hybridization of the first and second probe molecules to the nucleic acid molecule, such as via reaction between two reactive moieties to form a linking moiety. Alternatively, the first and second probe molecules may be linked to one another before the first and second probe molecules hybridize to the nucleic acid molecule.

All or a portion of the templated ligation processes described herein may be performed within a partition (e.g., as described herein). Alternatively, one or more such processes may be performed within a bulk solution. For example, one or more probe molecules may be subjected to conditions sufficient to hybridize to a nucleic acid molecule (e.g., a nucleic acid molecule included in an biological particle such as a cell) within a bulk solution. The nucleic acid molecule may be partitioned within various reagents (e.g., as described herein) including a nucleic acid barcode molecule, such as a nucleic acid barcode molecule releasably coupled to a bead (e.g., as described herein). Within the partition, the nucleic acid barcode molecule may hybridize to a sequence of a probe molecule hybridized to the nucleic acid molecule, thereby generated a barcode-linked nucleic acid molecule. Templated ligation processes may permit indirect barcoding of a nucleic acid molecule without the use of reverse transcription. Details of such processes and additional schemes are included in, for example, International Patent Publication No. WO2019/165318, which is herein entirely incorporated by reference for all purposes.

In some instances, barcoding of a nucleic acid molecule may be done using a combinatorial approach. In such instances, one or more nucleic acid molecules (which may be comprised in a biological particle, e.g., a cell, e.g., a fixed cell, organelle, nucleus, or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The barcoded nucleic acid molecules may thus comprise two barcode sequences (e.g., from the first nucleic acid barcode molecules and the second nucleic acid barcode molecules).

Additional barcode sequences may be attached to the barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 106 unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g. a sample or target nucleic acid molecule) in successive operations within a partition (e.g. droplet or well) to generate a barcoded nucleic acid molecule. A second unique nucleic acid molecule comprising one or more nucleic acid bases may be attached to the barcoded nucleic acid molecule molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety.

In some instances wherein a partition is a volume wherein diffusion of contents beyond the volume is inhibited, the partition contains a diffusion resistant material. Such partition may also be referred to herein as a diffusion resistant partition. The diffusion resistant material may have an increased viscosity. The diffusion resistant material may be or comprise a matrix, e.g., a polymeric matrix, or a gel. Suitable polymers or gels are disclosed herein. The matrix can be a porous matrix capable of entraining and/or retaining materials within its matrix. In some embodiments, a diffusion resistant partition comprises a single biological particle and a single bead, the single bead comprising a plurality of nucleic acid barcode molecules comprising a partition specific barcode sequence. In some embodiments the partition specific barcode sequence is unique to the diffusion resistant partition. In some embodiments, partitioning comprises contacting a plurality of biological particles with a plurality of beads in a diffusion resistant material to provide a diffusion resistant partition comprising a single biological particle and a single bead. In some embodiments, partitioning comprises contacting a plurality of biological particles with a plurality of beads in a liquid comprising a polymeric precursor material that may be capable of being formed into a gel or other solid or semi-solid matrix, and subjecting the liquid to conditions sufficient to polymerize or gel the precursors, e.g., as described herein. In some embodiments, the biological particle may be lysed or permeabilized in the diffusion resistant partition. In some embodiments, a nucleic acid analyte of the biological particle (which may include a reporter oligonucleotide associated with a labeling agent disclosed herein) may be coupled with a nucleic acid barcode molecule in the diffusion resistant partition. In some cases, further processing, e.g., generation of barcoded nucleic acid molecules, may be performed in the diffusion resistant partition or in bulk. For example, nucleic acid analytes, once coupled to nucleic acid barcode molecules in partitions, may be pooled and then subjected to further processing in bulk (e.g, extension, reverse transcription, or other processing) to generate barcoded nucleic acid molecules. For other example, nucleic acid analytes, one coupled to nucleic acid barcode molecules in diffusion resistant partitions, may be subjected to further processing in the diffusion resistant partitions to generate barcoded nucleic acid molecules.

In some cases, the target nucleic acid molecule is from a nucleic acid library prepared using a spatial analysis methodology described herein. Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a spatial capture probe which comprises a spatial barcode (e.g., a nucleic acid sequence that provides information as to the location or position of an analyte within a cell or a tissue sample, including a mammalian cell or a mammalian tissue sample) and a capture domain that is capable of binding to an analyte (e.g., a protein and/or a nucleic acid) produced by and/or present in a cell. Spatial analysis methods and compositions can also include the use of a spatial capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. The above references, if US patents or US Patent Publications, are incorporated herein by reference in their entirety. Further non-limiting aspects of spatial analysis methodologies and compositions are described herein.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, spatial capture probes may be configured to prime, replicate, and consequently yield optionally barcoded extension products from a template (e.g., a DNA or RNA template, such as an analyte or an intermediate agent, including a ligation product or an analyte capture agent, or a portion thereof), or derivatives thereof (see, e.g., Section (II)(b)(vii) of WO 2020/176788 and/or U.S. Patent Application Publication No. 2020/0277663 regarding extended spatial capture probes; incorporated herein by reference in their entirety). In some cases, spatial capture probes may be configured to form ligation products with a template (e.g., a DNA or RNA template, such as an analyte or an intermediate agent, or portion thereof), thereby creating ligations products that serve as proxies for a template.

As used herein, an “extended spatial capture probe” refers to a spatial capture probe having additional nucleotides added to the terminus (e.g., 3′ or 5′ end) of the spatial capture probe thereby extending the overall length of the spatial capture probe. For example, an “extended 3′ end” indicates additional nucleotides were added to the most 3′ nucleotide of the spatial capture probe to extend the length of the spatial capture probe, for example, by polymerization reactions used to extend nucleic acid molecules including templated polymerization catalyzed by a polymerase (e.g., a DNA polymerase or a reverse transcriptase). In some embodiments, extending the spatial capture probe includes adding to a 3′ end of a spatial capture probe a nucleic acid sequence that is complementary to a nucleic acid sequence of an analyte or intermediate agent specifically bound to the capture domain of the spatial capture probe. In some embodiments, the spatial capture probe is extended using reverse transcription. In some embodiments, the spatial capture probe is extended using one or more DNA polymerases. The extended spatial capture probes include the sequence of the spatial capture probe and the sequence of the spatial barcode of the spatial capture probe.

In some embodiments, extended spatial capture probes are amplified (e.g., in bulk solution or on the array) to yield quantities that are sufficient for downstream analysis, e.g., via DNA sequencing. In some embodiments, extended spatial capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of spatial capture probes, sequencing (e.g., of a cleaved extended spatial capture probe and/or a cDNA molecule complementary to an extended spatial capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663. The above references, if US patents or US Patent Publications, are incorporated herein by reference in their entirety.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

In some cases, spatial analysis can be performed by detecting multiple oligonucleotides that hybridize to an analyte. In some instances, for example, spatial analysis can be performed using RNA-templated ligation (RTL). Methods of RTL have been described previously (See, e.g., Credle et al., Nucleic Acids Res. 2017 Aug. 21; 45(14):e128). Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by spatial capture probes on an array, e.g., a spatially barcoded array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific spatial capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020). The above references, if US patents or US Patent Publications, are incorporated herein by reference in their entirety.

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320. The above references, if US patents or US Patent Publications, are incorporated herein by reference in their entirety.

Prior to transferring analytes from the biological sample to the array of features on the substrate, the biological sample can be aligned with the array. Alignment of a biological sample and an array of features including spatial capture probes can facilitate spatial analysis, which can be used to detect differences in analyte presence and/or level within different positions in the biological sample, for example, to generate a three-dimensional map of the analyte presence and/or level.

In some cases, a map of analyte presence and/or level can be aligned to an image of a biological sample using one or more fiducial markers (e.g., objects placed in the field of view of an imaging system which appear in the image produced, as described in the Substrate Attributes Section and Control Slide for Imaging Section of WO 2020/123320). Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Systems and methodologies in the field of spatial transcriptomics are designed to obtain spatially resolved analyte expression data (e.g., genomics, proteomics, transcriptomics) from tissues. In some examples, a tissue may be overlaid onto a support comprising barcoded oligonucleotides or spatial capture probes. Generally, the oligonucleotides comprise a spatial barcode, which is correlated with and is an identifier for the location of the particular oligonucleotide on the support (e.g., in some examples, oligonucleotides having known barcode sequences are printed onto designated areas of the support). When analytes are released from a biological sample and diffuse toward and contact the barcoded oligonucleotides, the barcoded oligonucleotides capture, or hybridize to, the analytes. In some examples, mRNAs may be the analytes and barcoded oligonucleotides may capture mRNAs having specific nucleotide sequences by hybridization, for example the barcoded oligonucleotides comprise a poly(T) capture domain that can hybridize a poly(A) tail of a mRNA. In the examples where mRNA is the analyte, reverse transcription of the captured mRNA can be initiated using added primers, and cDNA is produced using the barcoded oligonucleotide as a template. The resultant cDNA that is synthesized incorporates the barcodes included in the barcoded oligonucleotide. The cDNAs may be amplified. A library of the cDNAs/amplified cDNAs is prepared and nucleotide sequences of the libraries are obtained. Nucleotide sequences of the spatial barcodes provides for the data for an mRNA transcript to be mapped back to its location on the support, and by also obtaining an image of the tissue and cells overlaid onto the support at the beginning of the procedure, mRNA transcripts may be mapped to the location in the overlaid tissue, where the mRNA was expressed.

In some examples, a planar support on the surface of which is attached a spatially ordered arrangement of barcoded oligonucleotides comprising analyte capture domains is used. In some examples, an analyte capture domain may be an oligo(dT) sequence for capturing poly(A) sequences of eukaryotic mRNA. Other sequences may be used to capture specific nucleic acids, including specific mRNAs. The arrangement of the oligonucleotides on the surface of the support can be known because the oligonucleotides comprise spatial barcodes. In some examples, the oligonucleotides, with known spatial barcodes, are printed in a known pattern onto specific, known areas of the surface of the planar support in a predetermined arrangement. A tissue is then applied to the surface of the support and analytes (e.g., mRNA) are released from the cells that make up the tissue. mRNAs released from the tissue migrate to the surface of the support and hybridize to oligo(dT) capture domain sequences of the attached oligonucleotides. The hybridized mRNAs are amplified using reverse transcription into complementary oligonucleotides that include sequences from the captured mRNA linked to the spatial barcode of the oligonucleotide to which the mRNA bound. Obtaining and decoding the nucleotide sequences of the complementary oligonucleotides reveals where on the support specific mRNAs bound to oligonucleotides. These locations are then correlated to regions of the tissue that was applied to the surface of the support.

In modifications of the above method, a tissue sample may be probed for expression of specific proteins using antibodies. The antibodies may have attached nucleotide tags having a specific nucleotide sequence that capture domains of the barcoded molecules on a support are designed to capture through hybridization. Thus, proteomic data can be obtained from the oligonucleotide arrays.

FIG. 19 is a schematic diagram showing an exemplary spatial capture probe, as described herein. As shown, the spatial capture probe 102 is optionally coupled to a feature 101 by a cleavage domain 103, such as a disulfide linker. The spatial capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The spatial capture probe can also include a spatial barcode 105. The spatial capture probe can also include a unique molecular identifier (UMI) sequence 106. While FIG. 19 shows the spatial barcode 105 as being located upstream (5′) of UMI sequence 106, it is to be understood that spatial capture probes wherein UMI sequence 106 is located upstream (5′) of the spatial barcode 105 is also suitable for use in any of the methods described herein. The spatial capture probe can also include a capture domain 107 to facilitate capture of a target analyte. In some embodiments, the spatial capture probe comprises an additional functional sequence that can be located, e.g., between spatial barcode 105 and UMI sequence 106, between UMI sequence 106 and capture domain 107, or following capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a spatial capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a spatial capture probe attached to a given feature is different from the UMI sequence of a different spatial capture probe attached to the given feature.

Aspects of this disclosure include the following descriptions.

As used herein, the term “partition” may refer to a space or volume that may be suitable to contain one or more species or conduct one or more reactions. A partition may be a physical compartment, such as a droplet, a flowcell, a reaction chamber, a reaction compartment, a tube, a well, or a microwell. The partition may isolate space or volume from another space or volume. The droplet may be a first phase, e.g., an aqueous phase, or in a second phase, e.g., an oil, immiscible with the first phase. The droplet may be a first phase in a second phase that does not phase separate from the first phase, such as, for example, a capsule or liposome in an aqueous phase. A partition may comprise one or more inner partitions. In some cases, a partition may be a virtual compartment that can be defined and identified by an index, e.g., indexed libraries, across multiple and/or remote physical compartments. For example, a physical compartment may comprise a plurality of virtual compartments.

As used herein, the term “hybridization” or “hybridizes” may refer to the formation of a duplex between nucleotide sequences which are sufficiently complementary to form duplexes via Watson-Crick base pairing. Two nucleotide sequences can be “complementary” to one another when those molecules share base pair organization homology. “Complementary” nucleotide sequences can combine with specificity to form a stable duplex under appropriate hybridization conditions. For example, two sequences can be complementary when a section of a first sequence can bind to a section of a second sequence in an anti-parallel sense wherein the 3′-end of each sequence binds to the 5′-end of the other sequence and each A, T(U), G and C of one sequence is then aligned with a T(U), A, C and G, respectively, of the other sequence. RNA sequences can also include complementary G=U or U=G base pairs. Thus, two sequences need not have perfect homology to be “complementary.” Two sequences can be sufficiently complementary when at least about 80%, or at least about 90%, or at least about 95% of the nucleotides share base pair organization over a defined length of the molecule. Thus, the two sequences may have a number of mismatches.

Herein, “amplification product” refers to molecules that result from reproduction or copying of another molecule. Generally, the molecules copied or reproduced are nucleic acid molecules, specifically DNA or RNA molecules. In some examples, the molecule reproduced or copied may be used as a template for the produced molecules. In some examples, an analyte captured by the capture domain of an oligonucleotide may be used as a template to produce an amplification product. In some examples, an mRNA captured by the capture domain of an oligonucleotide may be used as a template to produce a cDNA amplification product. Various enzymes (e.g., reverse transcriptase) may be used for this process. The cDNA amplification product may in turn act as a template for amplification that may also be called amplification products. Various enzymes (e.g., Taq polymerase) may be used for this process.

Herein, “analyte” refers to a substance whose chemical constituents are being identified and/or measured. Generally, this application refers to analytes from and/or produced by cells. Any or all molecules or substances from or produced by a cell may be referred to herein as analytes. Chemically, cellular analytes may include proteins, polypeptides, peptides, saccharides, polysaccharides, lipids, nucleic acids, and other biomolecules. In some examples, analytes may be part of libraries.

Herein, “barcode,” generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). Barcodes can have a variety of different formats. For example, barcodes can include polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads. In some examples, a barcode may be a nucleotide sequence that is encoded by, linked to or associated with one or more oligonucleotides. In some examples, a specific barcode may correlate with a location of a barcode, on a support, for example. A barcode used to convey locational information may be called a spatial barcode.

Herein, “barcoded molecule” or, in some examples, “barcoded nucleic acid molecule” generally refers to a molecule or a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence (e.g., targeted by a primer sequence) or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule, e.g. mRNA.

Herein, “base-paired” generally refers to the situation where two complementary nucleic acids have formed hydrogen bonds between complementary nucleotides in the different strands. Two such nucleic acid strands may be referred to as hybridized to one another.

Herein, “capable” means having the ability or quality to do something.

Herein, “capture” generally refers to the capability of a first substance to interact with and/or bind a second substance where, for example, the second substance is part of a population of other substances (e.g., present in a library). An analyte may be captured. In some examples, capture refers to identification of a target nucleic acid molecule (e.g., an RNA) by its hybridization to a probe, and/or amplification of a target nucleic acid molecule or a nucleic acid probe hybridized to a probe (e.g., an RNA or a probe hybridized to the RNA) using, for example polymerase chain reaction (PCR) and/or nucleic acid extension of a target nucleic acid molecule or a probe hybridized to it using, for example reverse transcription reactions.

Herein, “capture domain” or “capture sequence” means a part of a molecule that is capable of binding or capturing a substance. An analyte capture domain may be capable of capturing analytes that may include proteins, polypeptides, peptides, saccharides, polysaccharides, lipids, nucleic acids, and other biomolecules. In some examples, an analyte capture domain may be a nucleotide sequence capable of hybridizing to an analyte that contains a complementary nucleotide sequence.

In some examples, an analyte capture domain may contain modified nucleotides.

Herein, “complementary,” in the context of one sequence of nucleic acids being complementary to another sequence, refers to the ability of two strands of single-stranded nucleic acids to form hydrogen bonds between the two strands, along their length. A complementary strand of nucleic acids is generally made using another nucleic acid strand as a template. A first nucleotide that is capable of hybridizing to a second nucleotide sequence may be said to be a complement of the second nucleotide sequence.

Herein, “configured to” generally refers to a component of a system that can perform a certain function.

Herein, “contact” refers to physical touching of separate substances or objects. “Contacting” refers to causing separate substances to physically touch one another.

Herein, “gel” means a semisolid colloidal suspension of a solid dispersed in a liquid. A gel is a type of medium. Types of gel may include agar, agarose, hydrogel, polyacrylamide and the like.

Herein, “generate” means to make or produce. Generally, herein, generate is used to describe producing complementary nucleic acid molecules (e.g., making an amplification product) using a template nucleic acid molecule.

As used herein, an affinity group may comprise two or more molecules that bind to each other, e.g., by forces other than those of covalent bonds. Some molecules of an affinity group may bind by covalent binding. An affinity group interaction describes the interaction between two or more molecules of the affinity group. An example affinity group is biotin-streptavidin.

Herein, “hybrid capture” refers to methods used to identify and/or isolate molecules (e.g., nucleic acids) based, at least in part, on their ability to hybridize to another nucleic acid molecule.

Herein, “hybridize” refers to a nucleotide sequence of a single-stranded nucleic acid molecule forming a complex with a nucleic acid molecule having a complementary nucleotide sequence. Generally, the complex forms through hydrogen bonding between complementary nucleotide bases in separate nucleic acid molecules.

Herein, “hybridizing nucleotide sequence” refers to a nucleotide sequence, within an oligonucleotide for example, that is capable of hybridizing with a complementary nucleotide sequence in a target nucleic acid molecule present on or within a cell from a tissue sample (e.g., cellular RNA). When a hybridizing nucleotide sequence is of such a length that it hybridizes with a complementary, either fully or partially, nucleotide sequence that is unique to a target nucleic acid molecule(s) (e.g., cellular RNA or family of RNAs), the hybridizing nucleotide sequence may be said to hybridize to the same target nucleic acid molecule (e.g., the same RNA).

Herein, “hydrogel” means a gel in which the liquid component is water.

Herein, “library” refers to a collection of molecules. In some embodiments, the molecules in a library may have nucleotide sequences that are generally representative (e.g., comprising the same nucleotide sequences or complementary nucleotide sequences) of nucleotide sequences present in the molecules from target nucleic acids. Molecules from which a library is made may act as templates for synthesis of the collection of molecules that make up the library. The “library” may be, or may be produced from, amplification products of target nucleic acids. Libraries may be created from amplification of cDNA that is made from mRNA analytes captured. In some embodiments, analytes may be captured on an array. A library may be derived from some captured target nucleic acids. In certain embodiments, a library may be derived from a sequencing process, such as next generation sequencing library preparation. In some aspects, a library may comprise barcoded molecules according to the methods described herein. In some embodiments, the barcoded molecules may comprise one or more functional sequences, for example, for processing by a sequencer, e.g., for attachment to a sequencing flow cell.

Herein, “nucleic acid” refers to linear macromolecules formed from polymerization of units called nucleotides.

Herein, “nucleotide” refers to a nucleoside linked to a phosphate group. Nucleotides are the basic structural units of nucleic acids like DNA.

Herein, “nucleotide sequence” refers to a linear progression of nucleotides within a nucleic acid molecule (e.g., oligonucleotide).

Herein, “oligonucleotide” means a linear polymer of nucleotides, in some examples 2′-deoxyribonucleotides. Oligonucleotides are single stranded. Oligonucleotides can be of various lengths. Oligonucleotides can include modified nucleotides as known in the art.

Herein, “permeable” refers to something that allows certain materials to pass through it. “Permeable” may be used to describe a cell in which analytes in the cell can leave the cell. “Permeabilize” is an action taken to cause, for example, a cell to release its analytes. In some examples, permeabilization of a cell is accomplished by affecting the integrity of a cell membrane such as by application of a protease or other enzyme capable of disturbing a cell membrane allowing analytes to diffuse out of the cell.

Herein, “primer” means a single-stranded nucleic acid sequence that provides a starting point for DNA synthesis. Generally, a primer has a nucleotide sequence that is complementary to a template, and has an available 3′-hydroxyl group to which a transcriptase or polymerase can add additional nucleotides complementary to corresponding nucleotides in the template, to synthesize a nucleic acid strand in the 3′ to 5′ direction.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

Herein, the term “sample” generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, tissue section, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

Herein, the terms “biological particle” generally refers to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix. A biological particle may comprise or be an biological particle.

Herein, the term “macromolecular constituent” generally refers to a macromolecule contained within or from an biological particle or biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

Herein, the term “molecular tag” generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

Herein, “section” generally refers to a thin layer or slice from a larger object.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by ILLUMINA, PACIFIC BIOSCIENCES, OXFORD NANOPORE, or LIFE TECHNOLOGIES. Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

Herein, “spatial” refers to a location within or on a space. In some examples, the space may be a two-dimensional space.

Herein, “species” generally refers to multiple oligonucleotides that have something in common. Generally, oligonucleotides considered to be part of the same species have at least one barcode in common. In some examples, the common barcode may be associated with a particular or a group of capture domain(s). In some examples, the common barcode may be associated with oligonucleotides on a support, for example, that are in proximity to one another. In some examples, the common barcode may be encoded by the oligonucleotides that are part of an array spot.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses). The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

Herein, “surface” means the outside part or upper layer of something. Herein, a “surface” of an array generally refers to a surface of a support or substrate that has oligonucleotides attached.

Herein, “template” refers to one single-stranded nucleic acid acting as a “template” for synthesis of another complementary single-stranded nucleic acid. For example, RNA can act as a template for synthesis of a complementary DNA strand synthesized using reverse transcriptase. A single-stranded DNA can act as a template for synthesis of a complementary DNA strand, most often by a DNA polymerase.

Herein, “unique” means one of a kind or unlike something else. In some examples, a “unique” mRNA may refer to an mRNA encoded by a single-copy gene.

Headings, e.g., (a), (b), (i) etc., are presented herein merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

As used herein, terms in singular form, for example, “a,” “an,” and “the” include the plural. For example, the terms “a” and “an” refer to “one or more.”

Unless defined otherwise, all technical and scientific terms used herein have the same meaning as understood by one of ordinary skill in the art to which the present invention pertains. It is to be understood that the terminology used herein is for describing particular embodiments only and is not intended to be limiting. In this disclosure, a term used in the singular form will also include the plural form and vice versa.

Embodiments of the disclosed inventions are illustrated in the accompanying drawings. It will be appreciated that the embodiments illustrated in the drawings are shown for purposes of illustration and not for limitation. It will be appreciated that changes, modifications and deviations from the embodiments illustrated in the drawings may be made without departing from the spirit and scope of the invention.

### EXAMPLES

### Example 1

Barcoded gene expression sequencing libraries are constructed by processing a suspension of single cells using the 10× Genomics Chromium Next GEM Single Cell 3′ Kit v 3.1 according to manufacturer's instructions. Additional information in this regard can be found at, e.g., https://www.10×genomics.com/support/single-cell-gene-expression/documentation/steps/library-prep/chromium-single-cell-3-reagent-kits-user-guide-v-3-1-chemistry-dual-index-with-feature-barcoding-technology-for-cell-surface-protein-and-cell-multiplexing.

The barcoded sequencing library is split into two aliquots.

Aliquot 1 is enriched for specific panels of gene targets using individually biotinylated (5′) probes using the 10× Genomics Targeted Gene Expression solution according to manufacturer's instructions. The biotinylated probes used to process Aliquot 1 are single stranded DNA oligonucleotides that target nucleic acid sequences of interest (such as by complementary base pairing). Each probe comprises a 5′ biotin modification. The probes are added to Aliquot 1 for hybridization, followed by the addition of streptavidin beads. The mix is then incubated to conjugate the biotinylated probes to the streptavidin beads. Subsequent washes remove non-hybridized library members. The remaining library molecules (that had hybridized to biotinylated probes and bound to the streptavidin beads) are amplified, e.g., with Illumina P5 and P7 primers, prior to sequencing.

Aliquot 2 is enriched for the same specific panels of gene targets according to the methods of the disclosure (see, e.g., FIGS. 1-10). By way of example only, primary probes are added to Aliquot 2 for hybridization, followed by a wash step to remove unhybridized probes. In some cases, the primary probes are comprise the same target-specific sequences as the probes used to process Aliquot 1, do not include a biotin modification, and further comprise universal capture sequences. In other cases, pairs of primary probes are used for each target sequence, such that the pairs hybridize to adjacent or nearly adjacent sequences of the target. Secondary probes (comprising universal sequences that are complementary to the univeral capture sequences of the unbiotinylated probes and further comprising a biotin modification) are added and allowed to hybridize to the remaining primary probes, followed by wash and subsequent addition of streptavidin beads. The library molecules (that remain associated with the streptavidin beads via hybridization of the primary probes to the secondary probes) are amplified, e.g., with Illumina P5 and P7 primers, prior to sequencing.

It is predicted that processing the library according to the methods of the disclosure (e.g., Aliquot 2) result in significant cost savings because only one universal biotinylated probe is needed to enrich libraries for targets of interest. In the case of libaries processed according to the methods described in FIGS. 3-10, that greater accuracy is obtained in the sequencing readout as compared to Aliquot 1, because of the use of two target-specific probes to hybridize to each target in proximity.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

1.-46. (canceled)

47. A method for processing a nucleic acid target molecule in a mixture of nucleic acid molecules, the method comprising:
contacting the mixture with a primary oligonucleotide which hybridizes to the nucleic acid target molecule, wherein the primary oligonucleotide comprises a target analyte capture sequence and a universal capture sequence;
contacting the mixture with a secondary oligonucleotide, the secondary oligonucleotide hybridizing to the primary oligonucleotide, wherein the secondary oligonucleotide comprises a universal sequence, the universal sequence hybridizing to the universal capture sequence of the primary oligonucleotide.

48. The method of claim 47, further comprising isolating the secondary oligonucleotides, which are attached to the primary oligonucleotides and the nucleic acid target molecules, using one or more bait molecules attached to each of the secondary oligonucleotides, the bait molecules having binding affinity to one or more binding partners.

49. The method of claim 48, further comprising removing nucleic acid molecules from the mixture that are not attached to the primary oligonucleotide.

50. The method of claim 47, wherein the mixture comprises barcoded nucleic acids.

51. The method of claim 47, wherein the nucleic acid target molecule is barcoded with one or more barcode sequences.

52. The method of claim 47, wherein the nucleic acid target molecule comprises a UMI sequence or at least a portion of a functional sequence.

53. The method of claim 47, wherein the universal capture sequence comprises a promoter.

54. The method of claim 47, wherein the universal capture sequence comprises a locked nucleic acid monomer or a chemically-modified nucleotide

55. The method of claim 54, wherein the universal capture sequence comprises a chemically-modified 2′-OMe nucleotide.

56. The method of claim 47, further comprising contacting the mixture with a plurality of primary oligonucleotides, wherein each primary oligonucleotide of the plurality of primary oligonucleotides comprises a common universal capture sequence.

57. The method of claim 47, wherein the primary oligonucleotide comprises two or more different universal capture sequences.

58. The method of claim 47, wherein the secondary oligonucleotide comprises a plurality of universal sequences that are identical.

59. The method of claim 47, further comprising contacting the mixture with a plurality of secondary oligonucleotides, wherein secondary oligonucleotide of the plurality of secondary oligonucleotides comprise a plurality of different universal sequences.

60. The method of claim 47, further comprising contacting the mixture with a plurality of primary oligonucleotides, wherein primary oligonucleotides of the plurality of primary oligonucleotides hybridize to a plurality of different target analyte sequences.

61. The method of claim 47, wherein the secondary oligonucleotide comprises an attached bait molecule having binding affinity to a binding partner.

62. The method of claim 61, wherein the binding partner is attached to a solid support.

63. The method of claim 62, wherein the solid support comprises a bead, a magnetic bead, or a paramagnetic bead

64. The method of claim 62, further comprising isolating the solid support.

65. The method of claim 61, wherein the bait molecule has specific binding affinity for the binding partner.

66. The method of claim 61, wherein the bait molecule is biotin and wherein the binding partner is streptavidin.
